(19) United States
(12) Patent Application Publication
(10) Pub. No.: US 2010/0049273 A1
(43) Pub. Date: Feb. 25, 2010

(54) USE OF LATE PASSAGE MESENCHYMAL STEM CELLS (MSCS) FOR TREATMENT OF CARDIAC RHYTHM DISORDERS

(76) Inventors: Glenn Gaudette, Holden, MA (US); Irina A. Potapova, East Setauket, NY (US); Peter R. Brink, Setauket, NY (US); Ira S. Cohen, Stony Brook, NY (US); Richard B. Robinson, Cresskill, NY (US); Michael R. Rosen, New York, NY (US)

Correspondence Address: KENYON & KENYON LLP, ONE BROADWAY, NEW YORK, NY, 10004, US

(21) Appl. No.: 12/374,030

(22) PCT Filed: Jul. 20, 2007
(86) PCT No.: PCT/US2007/016430

§ 371 Date: Oct. 19, 2009

Related U.S. Application Data
(60) Provisional application No. 60/832,518, filed on Jul. 21, 2006.

(51) Int. Cl. A61N 1/02; C12N 5/071; A61K 45/00; A61P 9/00

(52) U.S. Cl. 607/35; 435/366; 424/93.7; 607/9

(57) ABSTRACT

The present invention provides methods and compositions relating to the use of late passage mesenchymal stem cells (MSCs) for treatment of cardiac rhythm disorders. The late passage MSCs of the invention may be used to provide biological pacemaker activity and/or provide a bypass bridge in the heart of a subject afflicted with a cardiac rhythm disorder. The biological pacemaker activity and/or bypass bridge may be provided to the subject either alone or in tandem with an electronic pacemaker.

This research was supported by USPHS-NHLBI grants HL-28958 and HL-67101. The United States Government may have rights in this invention.

### INTRODUCTION

The present invention provides methods and compositions relating to the use of late passage mesenchymal stem cells (MSCs) for treatment of cardiac rhythm disorders. The late passage MSCs of the invention may be used to provide biological pacemaker activity and/or provide a bypass bridge in the heart of a subject afflicted with a cardiac rhythm disorder. The biological pacemaker activity and/or bypass bridge may be provided to the subject either alone or in tandem with an electronic pacemaker. The invention is based on the discovery that late passage MSCs have lost their ability to differentiate into cells of osteogenic, chondrogenic or adipogenic lineages, thereby enhancing their safety and efficacy.

### BACKGROUND OF INVENTION

Heart failure is a notoriously progressive disease, despite medical management. The increasing gap between the incidence of end-stage heart failure and surgical treatment is due, in great part, to the shortage of donor organs. Thus, there is a need for alternative approaches for treatment of damaged heart tissue that is not dependent of the availability of donor organs.

Although mesenchymal stem cells can be used as a vehicle for gene delivery to the cardiac syncytium, one significant drawback to the use of such cells is their ability to differentiate into different cell types of osteogenic, chondrogenic or adipogenic lineages. The present invention is based on the discovery that late passage MSCs have lost their ability to differentiate along different lineages thus increasing safety and efficacy. Accordingly, the present invention provides novel methods and compositions for treatment of cardiac disorders based on the use of late passage MSCs.

### SUMMARY OF THE INVENTION

The present invention provides methods and compositions relating to the use of late passage MSCs for treatment of cardiac disorders. The invention is based on the discovery that late passage MSCs have lost their ability to differentiate into cells of osteogenic, chondrogenic or adipogenic lineages, thereby enhancing their safety and efficacy.

Accordingly, the present invention relates to compositions comprising late passage MSCs that are substantially incapable of differentiation. In a preferred embodiment, the late passage MSCs have been passaged at least nine times. Additionally, the late passage MSCs of the invention express CD29, CD44, CD54 and HLA class I surface markers while failing to express CD14, CD45, CD34 and HLA class II surface markers.

In yet another embodiment of the invention, late passage MSCs may be genetically engineered to express a protein or oligonucleotide of interest. Such proteins or oligonucleotides may be those capable of providing biological pacemaker activity.

In a specific embodiment of the invention, the late passage MSCs are engineered to functionally expresses a hyperpolarization-activated, cyclic nucleotide-gated (HCN) ion channel, and wherein expression of the HCN channel is effective to induce a pacemaker current in said cell. In an embodiment of the invention, the expressed HCN channel is a mutant or chimeric HCN channel. Chimeric HCN channels are those HCN channels comprising an amino terminal portion, an intramembrane portion, and a carboxy terminal portion, wherein the portions are derived from more than one HCN isoform. In a preferred embodiment of the invention, the chimeric or mutant HCN channel provides an improved characteristic, as compared to a wild-type HCN channel, selected from the group consisting of faster kinetics, more positive activation, increased levels of expression, increased stability, enhanced cyclic nucleotide responsiveness, and enhanced neurohumoral response. Such late passage MSCs may also be engineered to functionally expresses a MiRP1 beta subunit along with an HCN channel.

In addition, this invention provides a biological pacemaker comprising a late passage MSC which functionally expresses an HCN ion channel or a mutant or chimera thereof, with or without a MiRP1 beta subunit or a mutant thereof, at a level effective to induce a pacemaker activity in the cell when implanted into a subject.

The present invention further relates to a pharmaceutical composition comprising a population of late passage MSCs, substantially incapable of differentiation, and a pharmaceutically acceptable carrier.

The present invention further provides a bypass bridge comprising gap junction-coupled late passage MSCs, which are substantially incapable of differentiation, the bridge having a first end and a second end, both ends capable of being attached to two selected sites in a heart, so as to allow the propagation of an electrical signal across the tract between the two sites in the heart. In a specific embodiment of the invention, the first end is capable of being attached to the atrium and the second end capable of being attached to the ventricle, so as to allow propagation of a pacemaker and/or electrical current/signal from the atrium to travel across the tract to excite the ventricle.

In yet another embodiment of the invention, the cells of the bypass tract functionally express at least one protein selected from the group consisting of: a cardiac connexin; an alpha subunit and accessory subunits of a L-type calcium channel; an alpha subunit with or without the accessory subunits of a sodium channel; and a L-type calcium and/or sodium channel in combination with the alpha subunit of a potassium channel, with or without the accessory subunits of the potassium channel.

In another embodiment of the invention, the cells of the bypass bridge functionally expresses: (i) a hyperpolarization-activated, cyclic nucleotide-gated (HCN) ion channel capable of generating a pacemaker current in said cell, (ii) a chimeric HCN channel comprising an amino terminal portion, an intramembrane portion, and a carboxy terminal portion, wherein the portions are derived from more than one HCN isoform, and wherein the expressed chimeric HCN channel generates a pacemaker current in said cell, or (c) a mutant HCN channel wherein the mutant HCN channel generates a pacemaker current in said cell.

Further, the present invention provides the use of the MSCs in a tandem pacemaker system comprising (1) an electronic pacemaker; (2) a biological pacemaker comprising an implantable late passage MSC that functionally expresses (a) an HCN ion channel, or (b) a chimeric HCN channel, or (c) a mutant HCN channel wherein the expressed HCN, chimeric HCN or mutant HCN channel generates an effective pacemaker current when said cell is implanted into a subject's heart; (3) and/or a bypass bridge comprising a strip of gap junction-coupled late passage MSCs having a first end and a second end, both ends capable of being attached to two selected sites in a heart, so as to allow the transmission of a pacemaker and/or electrical signal/current across the tract between the two sites in the heart. In an embodiment of the invention, the biological pacemaker of the tandem system, comprises at least about 5,000 late passage MSCs. In another embodiment of the invention, the biological pacemaker comprises at least about 200,000 late passage MSCs. In another embodiment of the invention, the tandem pacemaker system comprises at least about 700,000 late passage MSCs.

### BRIEF DESCRIPTION OF THE FIGURES

FIG. 1. Fat vacuoles in 4^{th} passage hMSCs exposed to adipogenic differentiation.

FIG. 2. 4^{th} passage hMSCs were first transfected with the PIRES-HCN2 plasmid followed by exposure to adipogenic differentiation. There are fewer cells with fat vacuoles but staining with oil red O still demonstrates a significant number of positive (red) cells.

FIG. 3. Minimal adiopogenic differentiation of 9^{th} passage non-transfected hMSCs is demonstrated by the presence of few fat vacuoles.

FIG. 4. Absence of adipogenic differentiation in 9^{th} passages hMSCs transfected with the PIRES-HCN2 plasmid.

FIG. 5. Western blots demonstrating abundant connexin 43 expression in 3^{rd} and 8^{th} passage hMSCs (right panel) and 3, 5 and 9^{th} passage hMSCs and 2^{nd} passage canine hMSCs (right panel).

FIG. 6. Caspase activation assay for apoptotic cells. Minimal activation is observed for hMSCs at passages 3, 5 or 10 indicating no predisposition to apoptosis.

FIG. 7. DNA analysis by gel electrophoresis of passages 2, 3 and 9 hMSCs. There is no DNA fragmentation, indicating that these passage hMSCs do not have a predisposition to apoptosis.

FIG. 8. Phenotypic characterization of hMSCs of passage 5 and 10 by flow cytometry demonstrating the presence of CD44 and CD54 antigen in both sets of cells.

FIG. 9 Phenotypic characterization of hMSCs of passages 5 and 10 by flow cytometry; HLA class I markers but not HLA class II markers are present on both sets of cells.

FIG. 10. Phenotypic characterization of hMSCs of passage 5 and 10 by flow cytometry; there is CD29 but not CD34 antigen in both sets of cells.

FIG. 11. Phenotypic characterization of hMSCs of passage 5 and 10 by flow cytometry; CD14 and CD45 antigens are absent in both sets of cells.

FIG. 12. Expression of HCN2-induced I_{f} like current is the same in cells from passages 5 and 9 transfected with the PIRES-HCN2 plasmid: FIG. 12A. Fluorescence images of passage 5 cells (upper two panels) and sample current record from patch clamp recordings (lower panel); FIG. 12B. Fluorescence images of passage 9 cells (upper 2 panels) and sample current record from patch clamp recordings (lower panel); FIG. 12C. Histogram comparing the capacitance (left 2 bars) and the HCN2-induced current density (right two bars). There is no significant difference in either parameter between hMSCs from passage 5 and 9.

FIG. 13. Biophysical properties of passage 5 and passage 9 cells expressing HCN2-induced current are very similar. FIG. 13A. Comparison of current records of HCN2-included current in passage 5 (left panel) and passage 9 (right panel) hMSCs. The current records are very similar. FIG. 13B. Activation curves obtained from passage 5 (left panel) and passage 9 (right panel) cells show the same midpoint of activation.

FIG. 14. Alignment of mammalian HCN1 polypeptide sequences. The mouse (SEQ ID NO:9), rat (SEQ ID NO:10), human (SEQ ID NO:11), rabbit (SEQ ID NO:12) and guinea pig (partial sequence; SEQ ID NO:13) HCN1 polypeptide sequences are aligned for maximum correspondence.

FIG. 15. Amino acid sequence of the human HCN212 chimeric channel. The shaded N-terminal portion of the sequence is derived from hHCN2; the underlined intramembranous portion from hHCN1; and the C-terminal portion (without shading or underline) from hHCN2. The amino acid sequence of the hHCN212 chimeric channel is set forth in SEQ ID NO:2. This 889-amino acid long chimeric hHCN212 sequence shows 91.2% identity with the 863-amino acid long mHCN212 sequence in 893 residues overlap when aligned for maximum correspondence.

FIG. 16. Amino acid sequence of the mouse HCN212 chimeric channel. The shaded N-terminal portion of the sequence is derived from mouse HCN2; the underlined intramembranous portion from mouse HCN1; and the C-terminal portion (without shading or underline) from mouse HCN2. The amino acid sequence of the mouse HCN212 chimeric channel is set forth in SEQ ID NO:6. This 863-amino acid long chimeric mHCN212 sequence shows 91.2% identity with the 889-amino acid long hHCN212 sequence in 893 residues overlap when aligned for maximum correspondence.

FIG. 17. Alignment of mammalian HCN2 polypeptide sequences. The mouse (SEQ ID NO:14), rat (SEQ ID NO:15), human (SEQ ID NO:16) and dog (partial sequence; SEQ ID NO:17) HCN2 polypeptide sequences are aligned for maximum correspondence.

FIG. 18. Alignment of mammalian HCN4 polypeptide sequences. The mouse (SEQ ID NO:18), rat (SEQ ID NO:19), human (SEQ ID NO:20), rabbit (SEQ ID NO:21) and dog (partial sequence; SEQ ID NO:22) HCN4 polypeptide sequences are aligned for maximum correspondence.

### DETAILED DESCRIPTION OF THE INVENTION

The present invention provides methods and compositions relating to the use of late passage mesenchymal late passage MSCs (MSCs) for treatment of cardiac rhythm disorders. The methods and compositions of the invention may be used in the treatment of cardiac disorders including, but not limited to, arrhythmias, myocardial dysfunction or infarction. Late passage MSCs may be genetically engineered to express one or more genes encoding physiologically active proteins of interest. Such proteins include, for example, proteins capable of providing biological pacemaker activity such as wild type, mutant and chimeric HCN ion channels and the HCN beta subunit MiRPI. In yet another embodiment of the invention late passage MSCs can be used to provide a bypass bridge to those subjects afflicted with sinoatrial or atrioventricular node disorders. The use of biological pacemakers and bypass bridges may be administered to a subject in need of pacemaker function either alone or in tandem with an electronic pacemaker.

### Late Passage MSCs

The present invention relates to methods and compositions relating to the use of late passage MSCs, which are substantially unable to differentiate, for treatment of cardiac disorders. As used herein, “late passage MSCs” are those cells that have been passaged at least nine times. Additionally, the late passage MSCs of the invention express CD29, CD44, CD54 and HLA class I surface markers while failing to express CD14, CD45, CD34 and HLA class II surface markers. In an embodiment of the invention, the late passage MSCs are mammalian in origin. In a preferred embodiment of the invention, the MSCs are derived from a human adult. Substantially unable to differentiate means that virtually all cells in a particular culture will not be able to differentiate. hMSCs that have been passaged at least nine times and that express CD29, CD44, CD54 and HLA class I surface markers while failing to express CD14, CD45, CD34 and HLA class II surface markers are considered “substantially not able to differentiate” as virtually, if not all, cells failed to differentiate into cells of osteogenic, chondrogenic or adipogenic lineages.

Human MSCs (Poietics™ hMSCs) to be used in the practice of the invention can be purchased from any reputable supplier such as Clonetics/Bio Whittaker (Walkersville, M.D.). Alternatively, late passage MSCs may be derived from bone marrow aspirates from the subject or from a healthy volunteer. For example, 10 ml of marrow aspirate is collected into a syringe containing 6000 units of heparin to prevent clotting, washed twice in phosphate buffer solution (PBS), added to 20 ml of control medium (DMEM containing 10% FBS), and then centrifuged to pellet the cells and remove the fat. The cell pellet is then resuspended in control medium and fractionated at 1100 g for 30 min on a density gradient generated by centrifugation of a 70% percoll solution at 13000 g for 20 minutes. The mesenchymal stem cell-enriched, low density fraction is collected, rinsed with control medium and plated at a density of 107 nucleated cells per 60 mm² dish. The mesenchymal late passage MSCs are then cultured in control medium at 37° C. in a humidified atmosphere containing 5% CO₂. A preferred culturing medium is a medium that prevents/inhibits differentiation, such as a medium sold by Cambrex Corporation, referred to as MSCGM medium.

Furthermore, antibodies that bind to cell surface markers selectively expressed on the surface of late passage MSCs may be used to identify or enrich for populations of MSCs using a variety of different methods. Such markers include, for example, CD29, CD44 and CD54 which are expressed on the surface of late passage MSCs.

The advantages to using MSCs are that they do not require an endoderm for differentiation, are easy to culture, do not require an expensive cytokine supplement and have minimal immunogenicity. The advantages to using late passage hMSCs is that they have lost their ability to differentiate into osteogenic, chondrogenic or adipogenic lineages thereby enhancing their efficacy and safety.

The present invention further provides pharmaceutical compositions comprising late passage MSCs and a pharmaceutically acceptable carrier. Pharmaceutically acceptable carriers are well known to those skilled in the art and include, but are not limited to, 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Preservatives and other additives, such as, for example, antimicrobials, antioxidants and chelating agents may also be included with all the above carriers.

### Use of Late Passage Human Mesenchymal Cells for Generation of Biological Pacemaker Activity

The present invention relates to the generation of biological pacemaker activity based on the expression of wild type, mutant or chimeric HCN ion channels in late passage MSCs for treatment of cardiac disorders. Methods for generating biological pacemaker activity are disclosed in U.S. Pat. No. 6,849,611 and U.S. patent application Ser. Nos. 10/342,506 and 10/757,827 each of which are incorporated by reference herein in their entirety.

As used herein, “biological pacemaker activity” shall mean the rhythmic generation of an action potential originating from the introduction of biological material in a cell or a syncytial structure comprising the cell. A “syncytial structure” shall mean a structure with gap junction-mediated communication between its cells.

The present invention relates to the generation of biological pacemakers with desirable clinical characteristics based on late passage MSCs expression of wild-type, mutant and chimeric HCN genes, and the use of these biological pacemakers to create an effective treatment for cardiac conditions. Accordingly, the present invention provides late passage hMCSs comprising in vitro-recombined gene constructs encoding HCN ion channels. An “HCN ion channel” shall mean a hyperpolarization-activated, cyclic nucleotide-gated ion channel responsible for the hyperpolarization-activated cation currents that are directly regulated by cAMP and contribute to pacemaker activity in heart and brain. “mHCN” designates murine or mouse HCN; “hHCN” designates human HCN.

There are four HCN isoforms: HCN1, HCN2, HCN3 and HCN4. All four isoforms are expressed in brain; HCN1, HCN2 and HCN4 are also prominently expressed in heart, with HCN4 and HCN1 predominating in sinoatrial node and HCN2 in the ventricle.

In an embodiment of the invention, the HCN channel to be expressed is HCN1, HCN2, HCN3, HCN4, or a mutant thereof. Voltage sensing and activation of HCN channels can be altered by mutation. For example, Chen et al. (2001, Proc. Natl. Acad. Sci USA-98:11277-11282) identified three residues, E324, Y331, and R339, in the mHCN2 S4-S5 linker that, when mutated, disrupts normal channel closing. Mutation of a basic residue in the S4 domain (R318Q) prevents channel opening. Conversely, channels with R318Q and Y331S double mutations are constitutively open. Several point mutations, including R318Q, W323A, E324A, E324D, E324K, E324Q, F327A, T330A and Y331A, Y331D, Y331F, Y331K, D332A, M338A, R339A, R339C, R339D, R339E and R339Q, were also made by Chen et al. (2001, Proc. Natl. Acad. Sci USA 98:11277-11282) to investigate in greater detail the role of the E324, Y331 and R339 residues in voltage sensing and activation. Many additional mutations in different HCN isoforms have been reported. For example, Chen et al. (2001, J Gen Physiol 117:491-504) have reported the R538E and R591E mutations in mHCN1; Tsang et al. (2004, J Biol Chem 279:43752-43759) have reported G231A and M232A in mHCN1; Vemana et al (2004, J Gen Physiol 123:21-32) have reported R247C, T249C, K250C, 1251C, L252C, S253C, L254C, L258C, R259C, L260C, S261C, C318S, S338C in mHCN2; Macri and Accili (2004, J Biol Chem 279:16832-16846) have reported S306Q, Y331D AND G404S in mHCN2; and Decher et al. (2004, J Biol Chem 279:13859-13865) have reported Y331A, Y331D, Y331S, R331FD, R339E, R339Q, 1439A, S441A, S441T, D443A, D443C, D443E, D443K, D443N, D443R, R447A, R447D, R447E, R447Y, Y449A, Y449D, Y449F, Y449G, Y449W, Y453A, Y453D, Y453F, Y453L, Y453W, P466Q, P466V, Y476A, Y477A and Y481A in mHCN2. The contents of all of the above publications are incorporated herein by reference. Certain of the reported mutations listed above may confer, singly or in combination, beneficial characteristics on the HCN channel with regard to creating a biological pacemaker. The invention disclosed herein encompasses late passage MSC expression of mutations in HCN channels, singly or in combinations, which enhance pacemaker activity of the channel. In a preferred embodiment, the HCN channel or mutant thereof is HCN2.

Mutations are identified herein by a designation with provides the single letter abbreviation of the amino acid residue that underwent mutation, the position of that residue within a polypeptide, and the single letter abbreviation of the amino acid residue to which the residue was mutated. Thus, for example, E324A identifies a mutant polypeptide in which the glutamate residue (E) at position 324 was mutated to alanine (A). Y331A, E324A-HCN2 indicates a mouse HCN2 having a double mutation, one in which tyrosine (Y) at position 331 was mutated to alanine (A), and the other in which the glutamate residue at position 324 was mutated to alanine.

In a specific embodiment of the present invention, the mutant HCN2 channel is E324A-HCN2, Y331A-HCN2, R339A-HCN2, or Y331A, E324A-HCN2. In a preferred embodiment, the mutant HCN2 channel is E324A-HCN2.

One approach to enhancing biological pacemaker activity of a HCN channel by increasing the magnitude of the current expressed and/or speeding its kinetics of activation is to co-express with HCN2 its beta subunit, MiRP1. MiRP1 mutations have also been reported (see e.g., Mitcheson et al., (2000, J Gen Physiol 115:229-40); Lu et al., (2003, J Physiol 551:253-62); Piper et al., (2005, J Biol Chem 280:7206-17)), and certain of these mutations, or combinations thereof, may be beneficial in increasing the magnitude and kinetics of activation of the current expressed by a HCN channel used to create a biological pacemaker. The invention disclosed herein encompasses all such mutations, or combinations thereof, in MiRP1.

The present invention further relates to the use of late passage MSCs expressing chimeras between HCN isoforms for generating pacemaker currents in treating heart disorders. Such chimeric HCN channels may be formed by in vitro recombination of nucleotide sequences encoding portions of all four HCN isoforms to produce HCN chimeras. Chimeras of pacemaker ion channels that may be used in the practice of the invention include, but are not limited to, those chimera channels disclosed in U.S. Provisional Patent Application No. 60/715,934 and 60/832,515, filed Jul. 21, 2006, entitled “Chimeric HCN Channels,” which are both incorporated herein by reference in their entirety.

A “HCN chimera” shall mean an ion channel comprised of portions of more than one type of HCN channel. For example, a chimera of HCN1 and HCN2 or HCN3 or HCN4, and so forth. In an embodiment of the invention, the portions are derived from human HCN isoforms. In addition a chimera ion channel may also comprise portions of an HCN channel derived from different species. For example, one portion of the channel may be derived from a human and another portion may be derived from a non-human.

Such chimeric HCN polypeptides provide an improved characteristic, as compared to a wild-type HCN channel, selected from the group consisting of faster kinetics, more positive activation, increased expression, and/or increased stability, enhanced cAMP responsiveness, and enhanced neurohumoral response.

In general terms, HCN polypeptides can be divided into three major domains: (1) an amino terminal portion; (2) an intramembranous portion and its linking regions; and (3) a carboxy-terminal portion. Structure-function studies have shown that the intramembranous portions with its linking regions play an important role in determining the kinetics of gating. The C-terminal portion contains a binding site for cAMP and so is in large part responsible for the ability of the channel to respond to the sympathetic and parasympathetic nervous systems that respectively raise and lower cellular cAMP levels.

The term “HCNXYZ” (wherein X, Y and Z are any one of the integers 1, 2, 3 or 4, with the proviso that at least one of x, y and Z is a different number from at least one of the remaining) shall mean an HCN chimera channel polypeptide comprising three contiguous portions in the order XYZ wherein X is an N-terminal portion, Y is an intramembrane portion, and Z is a C-terminal portion, and wherein the number of X, Y and Z designates the HCN channel from which that portion is derived. For example, HCN112 is an HCN chimera with a N-terminal portion and intramembrane portion from HCN1 and a C-terminal portion from HCN2.

The present invention provides late passage hMCSs comprising in vitro-recombined gene constructs encoding chimeric HCN channels that have fast kinetics and good responsiveness to cAMP. In one embodiment of the invention described herein, the HCN chimera comprises an amino terminal portion contiguous with an intramembranous portion contiguous with a carboxy terminal portion, wherein each portion is a portion of an HCN channel or a portion of a mutant thereof, and wherein one portion derives from an HCN channel or a mutant thereof which is different from the HCN channel or mutant thereof from which at least one of the other two portions derive.

In a specific embodiment, the mutant HCN channel from which the portion of the HCN chimera derives is E324A-HCN2, Y331A-HCN2, R339A-HCN2, or Y331A, E324A-HCN2. In a still further embodiment, the HCN chimera is a polypeptide comprising mHCN112, mHCN212, mHCN312, mHCN412, mHCN114, mHCN214, mHCN314, mHCN414, hHCN112, hHCN212, hHCN312, hHCN412, hHCN114, hHCN214, hHCN314, or hHCN414. In a specific embodiment of the invention the chimeric HCN polypeptide is hHCN212 or polypeptide mHCN212.

Other preferred embodiments include: a chimeric HCN polypeptide wherein the intramembranous portion is derived from an HCN1 channel; a chimeric HCN polypeptide wherein the intramembranous portion is D140-L400 of hHCN1; or a chimeric HCN polypeptide wherein the intramembranous portion is D129-L389 of mHCN1.

In yet another embodiment of the invention, the chimeric HCN polypeptide is a mutant HCN channel containing a mutation in a region of the channel selected from the group consisting of the S4 voltage sensor, the S4-S5 linker, S5, S6 and S5-S6 linker, the C-linker, and the carboxy-terminal cyclic nucleotide binding domain (“CNBD”).

In yet another embodiment of the invention, the chimeric HCN polypeptide is a mutant, wherein the mutant portion is derived from mHCN2 having the sequence set forth in SEQ ID NO:14 and comprises E324A-mHCN2, Y331A-mHCN2, R339A-mHCN2, or Y331A, E324A-mHCN2. In a specific embodiment of the invention, the mutant portion comprises E324A-mHCN2.

In addition to recombinant expression of wild-type, mutant and chimeric HCN ion channels, the late passage MSCs may further expresses at least one cardiac connexin, including for example, Cx43, Cx40, or Cx45.

To practice the methods of the invention it will be necessary to recombinantly express wild-type, mutant and chimeric HCN ion channels. The cDNA sequence and deduced amino acid sequence of HCN ion channels have been characterized. Sequences of the HCN ion channels are available from public databases.

HCN ion channel nucleotide sequences may be isolated using a variety of different methods known to those skilled in the art. For example, a cDNA library constructed using RNA from a tissue known to express the HCN ion channels can be screened using a labeled HCN channel probe. Alternatively, a genomic library may be screened to derive nucleic acid molecules encoding the HCN ion channel protein. Further, such nucleic acid sequences may be derived by performing a polymerase chain reaction (PCR) using two oligonucleotide primers designed on the basis of known HCN ion channel nucleotide sequences. The template for the reaction may be cDNA obtained by reverse transcription of mRNA prepared from cell lines or tissue known to express the HCN ion channel of interest.

HCN ion channels, polypeptides and peptide fragments, mutated, truncated, deleted and chimeric forms of the HCN channels can be prepared for a variety of uses, including but not limited to, the production of biological pacemaker activity. Such proteins may be advantageously produced by recombinant DNA technology using techniques well known in the art for expressing a nucleic acid. Such methods can be used to construct expression vectors containing the HCN ion channel nucleotide sequences and appropriate transcriptional and translational control signals. These methods include, for example, in vitro recombinant DNA techniques, synthetic techniques, and in vivo genetic recombination. (See, for example, the techniques described in Sambrook J et al. 2000. Molecular Cloning: A Laboratory Manual (Third Edition), and Ausubel et al (1996) Current Protocols in Molecular Biology John Wiley and Sons Inc., USA).

A variety of host-expression vector systems maybe utilized to express the HCN ion channel nucleotide sequences in late passage MSCs. For long-term, high yield production of recombinant HCN ion channel expression, such as that desired for development of biological pacemakers, stable expression is preferred. Rather than using expression vectors which contain origins of replication, host cells can be transformed with DNA controlled by appropriate expression control elements and a selectable marker gene, i.e., tk, hgprt, dhfr, neo, and hygro gene, to name a few. Following the introduction of the foreign DNA, engineered late passage MSCs may be allowed to grow for 1-2 days in enriched media, and then switched to a selective media.

Any of the methods for gene delivery into a host cell available in the art can be used according to the present invention. Such methods include, for example, electroporation, lipofection, calcium phosphate mediated transfection, or viral infection. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

The present invention further provides compositions comprising MSCs expressing wild-type, mutant or chimera HCN channels, as described above. The compositions of the invention may further comprise a pharmaceutically acceptable carrier.

The present invention relates to a method of treating a subject afflicted with a cardiac rhythm disorder comprising administering a late passage MSC, expressing wild-type, mutant or chimeric HCN polypeptides, to a region of the subject's heart, wherein expression of the HCN polypeptide in said region of the heart is effective to induce a pacemaker current in the heart and thereby treat the subject. In a specific embodiment of the invention, the late passage MSC forms a functional syncytium with the heart.

In an embodiment of the invention, the late passage MSC, expressing wild-type, mutant or chimeric HCN polypeptides is administered to the region of the heart by injection, catheterization, surgical insertion, or surgical attachment. The late passage MSCs may be locally administered by injection or catheterization directly onto or into the heart tissue. The late passage MSCs may be administered by injection or catheterization into at least one of a coronary blood vessel or other blood vessel proximate to the heart. The late passage MSCs may administered to any suitable region of the heart, including, but not limited to, the Bachmanns bundle, sinoatrial node, atrioventricular junctional region, His branch, left or right atrial or ventricle muscle, left or right bundle branch, or Purkinje fibers.

Cardiac rhythm disorders that may be treated using the methods and compositions of the invention include, but are not limited to, sinus node dysfunction, sinus bradycardia, marginal pacemaker function, sick sinus syndrome, tachyarrhythmia, sinus node reentry tachycardia, atrial tachycardia from an ectopic focus, atrial flutter, atrial fibrillation, bradyarrhythmia, or cardiac failure, wherein the late passage MSCs expressing wild-type, mutant or chimeric HCN polypeptides, are administered to the right or left atrial muscle, sinoatrial node, or atrioventricular junctional region of the subject's heart.

Disorders to be treated also include a conduction block, complete atrioventricular block, incomplete atrioventricular block, or bundle branch block, wherein the late passage MSC, expressing wild-type, mutant or chimeric HCN polypeptides, are administered to a region of the subject's heart so as to compensate for the impaired conduction in the heart. Such regions include the ventricular septum or free wall, atrioventricular junctional region, or bundle branch of the ventricle.

The present invention additionally provides a method of inhibiting the onset of a cardiac rhythm disorder in a subject prone to such disorder comprising administering a late passage MSC, expressing wild-type, mutant or chimeric HCN polypeptides, to a region of the subject's heart, wherein expression of the HCN polypeptide in the heart is effective to induce a pacemaker current in the heart and thereby inhibit the onset of the disorder in the subject.

### Use of Late Passage Human Mesenchymal Stem Cells for Generation of a Bypass Bridge

The present invention also provides compositions for treating a subject afflicted with a cardiac rhythm disorder comprising providing a bypass bridge in the heart that will take over the function of a diseased atrioventricular or sinus node. Methods for production of such bypass bridges are disclosed in International Patent Application No. PCT/US04/042953 and U.S. application Ser. No. 11/490,760, filed Jul. 21, 2006, entitled “A Biological Bypass Bridge with Sodium Channels, Calcium Channels and/or Potassium Channels to Compensate for Conduction Block in the Heart,” which are both incorporated herein by reference in their entirety.

In an embodiment of the invention, the bypass bridge may be made from a strip of late passage hMSCs without incorporation of additional molecular determinants of conduction. Here the cells' own ability to generate gap junctions that communicate pacemaker and/or electrical currents/signals are used as a means to propagate an pacemaker and/or electrical wave from cell to cell.

Accordingly, the present invention provides a bypass bridge comprising a tract of gap junction-coupled late passage hMSCs having a first end and a second end, both ends capable of being attached to two selected sites in a heart so as to allow the conduction of an electrical signal across the tract between the two sites, wherein the cells functionally express a sodium channel. Such sodium channels include, for example, a SKM-1 channel which may further comprise an alpha subunit and/or an accessory subunit.

In a specific embodiment of the invention, the first end of the tract is capable of being attached to the atrium and the second end of the tract is capable of being attached to the ventricle, so as to allow conduction of an electrical signal across the tract from the atrium to the ventricle.

In an embodiment of the invention, the late passage MSCs of the bypass bridge may further functionally express a pacemaker ion channel which induces a pacemaker current so as to induce a pacemaker current in said cells. The pacemaker ion channel is at least one of (a) a hyperpolarization-activated, cyclic nucleotide-gated (HCN) ion channel, mutant or chimera thereof, with or without (b) a MiRP1 beta subunit. Mutants and chimeras HCN channels are described in detail above. In an embodiment of the invention, the pacemaker ion channel is expressed in cells in the first end of the tract. In a specific embodiment, the cells expressing the pacemaker ion channel are located in a region extending 0.5 mm from the first end.

The late passage MSCs in the tract may further functionally express one or more additional channels, including but not limited to, a potassium channel which may further comprise a Kir2.1 or Kir2.2 alpha subunit and/or an accessory subunit; and an L-type calcium channel which may further comprise an alpha subunit and an accessory subunit.

Thus, the cells of the bypass bridge may further functionally express one or more of at least one cardiac connexin, an alpha subunit with accessory subunits of an L-type calcium channel, an alpha subunit with or without accessory subunits of a potassium channel, so as to change the voltage-time course of repolarization and/or refractoriness of the heart. Connexins that may be expressed include, but are not limited to, Cx43, Cx40, or Cx45.

The present invention provides a method of making a bypass bridge for implantation in a heart comprising: (a) transfecting a late passage MSC with, and functionally expressing therein, a nucleic acid encoding a sodium channel; and (b) growing the transfected late passage MSC into a tract of cells having a first and a second end capable of being attached to two selected sites in the heart, wherein the cells are physically interconnected via electrically conductive gap junctions.

In an embodiment of the invention, cells in the tract are transfected with a nucleic acid encoding a pacemaker ion channel, wherein the nucleic acid is functionally expressed so as to induce a pacemaker current in the cells. The pacemaker ion channel is at least one of (a) a hyperpolarization-activated, cyclic nucleotide-gated (HCN) ion channel or a mutant or chimera thereof, with or without (b) a MiRP1 beta subunit.

The late passage MSCs may be further transfected with, at least one nucleic acid encoding one or more of at least one cardiac connexin, an alpha subunit with accessory subunits of an L-type calcium channel, an alpha subunit with or without accessory subunits of the potassium channel, such that implantation of a bypass bridge in a heart changes the voltage-time course of repolarization and/or refractoriness of the heart.

The present invention provides a method of implanting a bypass bridge in a heart comprising: (a) making a bypass bridge utilizing the methods of the present invention; (b) selecting a first and a second site in the heart; and (c) attaching the first end of the tract to a first site and the second end of the tract to a second site; so as to thereby implant a bypass bridge in the heart that allows the conduction of a pacemaker and/or electrical signal/current across the tract between the two sites. In an embodiment of the invention, the electrical signal is generated in the atrium by the sinus node or an electronic pacemaker.

The present invention further provides a method of treating a disorder associated with an impaired conduction in a subject's heart comprising: (a) transfecting a late passage MSC with a nucleic acid encoding a sodium channel, wherein the cell functionally expresses the sodium channel; (b) growing the transfected late passage MSC into a tract of cells having a first end and a second end, wherein the cells are physically interconnected via electrically conductive gap junctions; (c) selecting a first site and a second site in the heart between which sites conduction is impaired; and (d) attaching the first end of the tract to the first site and the second end of the tract to the second site; so as to allow the conduction of an electrical signal across the tract between the two sites and thereby treat the subject.

The present invention relates to a method of treating a disorder associated with an impaired conduction and impaired sinus node activity in a subject's heart comprising: (a) transfecting a late passage MSC with at least one nucleic acid encoding a sodium channel and a pacemaker ion channel, wherein the late passage MSC functionally expresses the sodium channel and the pacemaker ion channel; (b) growing the transfected late passage MSC into a tract of cells having a first end and a second end, wherein the cells are physically interconnected via electrically conductive gap junctions; (c) selecting a first site in the left atrium of the heart and a second site, between which sites conduction is impaired; and (d) attaching the first end of the tract to the first site and the second end of the tract to the second site; so as to allow the propagation of an electrical signal generated by the sinus node and/or tract of cells between the two sites and thereby treat the subject.

The preparation of a bypass bridge in this fashion not only facilitates propagation from atrium to ventricle, but provides sufficient delay from atrial to ventricular contraction to maximize ventricular filling and emptying to mimic the normal activation and contractile sequence of the heart. Moreover, this approach, when used with biological pacemaker technology to improve atrial impulse initiation in the setting of sinus node disease offers a completely physiologic system. Thus, the present methods comprise the use in a subject's heart of various combinations of a biological pacemaker and/or biological atrioventricular bridge or atrioventricular node.

### Use of Mesenchymal Stem Cells in Biological Pacemakers and/or Bypass Bridges in Tandem with Electronic Pacemakers

The present invention relates to the use of MSCs in biological pacemakers and/or bypass bridges either alone or in combination with electronic pacemakers. Detailed descriptions of the individual components of a tandem pacemaker have been previously published. For example, details of electronic pacemakers per se may be found in U.S. Pat. No. 5,983,138; U.S. Pat. No. 5,318,597; U.S. Pat. No. 5,376,106; Pacemaker Timing Cycles and Electrocardiography, David L. Hayes, M. D., Chapter 6 of Cardiac Pacing and Defibrillation, pp. 201-223, Mayo Foundation, 2000; and Types of Pacemakers and Hemodynamics of Pacing, Chapter 5 of A Practical Guide to Cardiac Pacing-Fifth Edition, pp. 78-84, Cippincott Williams & Wilkins, Philadelphia (2000) all of which are incorporated herein by reference. Additionally, tandem cardiac pacemakers to be used in combination with biological pacemakers and/or bypass bridges are described in U.S. Patent Application Ser. Nos. 60/701,312 (filed on Jul. 21, 2005) and 60/781,723 (filed on Mar. 14, 2005) and Ser. No. 11/490,997 (filed on Jul. 21, 2006), entitled “Tandem pacemaker systems” each of which are incorporated by reference herein in their entirety.

In preferred embodiments of the subject invention, the electronic pacemaker is programmed to produce its pacemaker signal on an “as-needed” basis, i.e., to sense the biologically generated beats and to discharge electrically when there has been failure of the biological pacemaker to fire and/or atrioventricular bridge to conduct an impulse for more than a preset time interval. At this point the electronic pacemaker will take over the pacemaker function until the biological pacemaker resumes activity and/or the atrioventricular bridge resumes impulse conduction. Accordingly, a determination should be made on when the electronic pacemaker will produce its pacemaker signal. State of the art pacemakers have the ability to detect when the heart rate falls below a threshold level in response to which an electronic pacemaker signal should be produced. The threshold level may be a fixed number, but preferably it varies depending on patient activity such as physical activity or emotional status. When the patient is at rest or pursuing light activity the patient's baseline heart rate may be at 50-80 beats per minute (bpm) (individualized for each patient), for example. Of course, this baseline heart rate varies depending on the age and physical condition of the patient, with athletic patients typically having lower baseline heart rates. The electronic pacemaker can be programmed to produce a pacemaker signal when the patient's actual heart rate (including that induced by any biological pacemaker) falls below a certain threshold baseline heart rate, a certain differential, or other ways known to those skilled in the art. When the patient is at rest the baseline heart rate will be the resting heart rate. The baseline heart rate will likely change depending on the physical activity level or emotional state of the patient. For example, if the baseline heart rate is 80 bpm, the electronic pacemaker may be set to produce a pacemaker signal when the actual heart rate is detected to be about 64 bpm (i.e., 80% of 80 bpm).

The electronic component can also be programmed to intervene at times of exercise if the biological component fails, by intervening at a higher heart rate and then gradually slowing to a baseline rate. For example, if the heart rate increases to 120 bpm due to physical activity or emotional state, the threshold may increase to 96 bpm (80% of 120 bpm). The biological portion of this therapy brings into play the autonomic responsiveness and range of heart rates that characterize biological pacemakers and the baseline rates that function as a safety-net, characterizing the electronic pacemaker. The electronic pacemaker may be arranged to output pacemaker signals whenever there is a pause of an interval of X % (e.g., 26%) greater than the previous interval, as long as the previous interval was not due to an electronic pacemaker signal and was of a rate greater than some minimum rate (e.g., 50 bpm).

In an embodiment of the present methods, the electronic pacemaker senses the heart beating rate and produces a pacemaker signal when the heart beating rate falls below a specified level. In a further embodiment, the specified level is a specified proportion of the beating rate experienced by the heart in a reference time interval. In a still further embodiment, the reference time interval is an immediately preceding time period of specified duration.

The present invention provides a tandem pacemaker system comprising (1) an electronic pacemaker, and (2) a biological pacemaker, wherein the biological pacemaker comprises an implantable late passage MSC that functionally expresses a wild type, mutant or chimeric hyperpolarization-activated, cyclic nucleotide-gated (HCN) ion channel, and wherein the expressed HCN channel generates an effective pacemaker current when the cell is implanted into a subject's heart. Wild type, mutant and chimeric HCN channel expression can be achieved using the methods described above.

In an embodiment of the invention, the biological pacemaker of the tandem system comprises at least about 5,000 late passage MSCs. In another embodiment of the invention, the biological pacemaker comprises at least about 200,000 late passage MSCs. In another embodiment of the invention, the biological pacemaker comprises at least about 700,000 late passage MSCs.

In a specific embodiment of the invention, a tandem pacemaker system is provided comprising (1) an electronic pacemaker, and (2) a biological pacemaker, wherein the biological pacemaker comprises an implantable late passage MSC, wherein said cell functionally expresses a chimeric HCN ion channel, wherein said chimeric HCN is hHCN212, and wherein the expressed chimeric HCN channel generates an effective pacemaker current when the cell is implanted into a subject's heart, and wherein the biological pacemaker comprises at least about 700,000 human adult mesenchymal late passage MSCs.

Further, the present invention provides a tandem pacemaker system comprising (1) an electronic pacemaker, and (2) a bypass bridge comprising a strip of gap junction-coupled late passage MSCs having a first end and a second end, both ends capable of being attached to two selected sites in a heart, so as to allow the transmission of a pacemaker and/or electrical signal/current across the tract between the two sites in the heart.

In a specific embodiment of the invention, the first end of the bypass bridge is capable of being attached to the atrium and the second end capable of being attached to the ventricle, so as to allow transmission of an electrical signal from the atrium to travel across the tract to excite the ventricle. Further, the late passage MSCs of the bypass bridge can functionally express at least one protein selected from the group consisting of: a cardiac connexin; an alpha subunit and accessory subunits of a L-type calcium channel; an alpha subunit with or without the accessory subunits of a sodium channel; and a L-type calcium and/or sodium channel in combination with the alpha subunit of a potassium channel, with or without the accessory subunits of the potassium channel. Such cardiac connexins are selected from the group consisting of Cx43, Cx40, and Cx45.

Further, the present invention provides a tandem pacemaker system comprising (1) an electronic pacemaker, (2) a bypass bridge comprising a strip of gap junction-coupled late passage MSCs having a first end and a second end, both ends capable of being attached to two selected sites in a heart, so as to allow the transmission of a pacemaker and/or electrical signal/current across the tract between the two sites in the heart, and (3) a biological pacemaker comprising comprises an implantable late passage MSC that functionally expresses a (a) an HCN ion channel, or (b) a chimeric HCN channel wherein the chimeric HCN channel comprises portions of more than one type of HCN channel, or (c) a mutant HCN channel wherein the expressed HCN, chimeric HCN or mutant HCN channel generates an effective pacemaker current when said cell is implanted into a subject's heart. In an embodiment of the invention, the biological pacemaker of the tandem system, comprises at least about 5,000 late passage MSCs. In another embodiment of the invention, the biological pacemaker comprises at least about 200,000 late passage MSCs. In another embodiment of the invention, the tandem pacemaker system comprises at least about 700,000 late passage MSCs.

The present invention provides a method of treating a subject afflicted with a cardiac rhythm disorder, which method comprises administering a tandem pacemaker system as described herein to the subject, wherein the biological pacemaker of the system is provided to the subject's heart to generate an effective biological pacemaker current and further providing the electronic pacemaker to the subject's heart to work in tandem with the biological pacemaker to treat the cardiac rhythm disorder. The electronic pacemaker may be provided before the biological pacemaker, simultaneously with the biological pacemaker or after the biological pacemaker. The biological pacemaker is designed to enhance beta-adrenergic responsiveness of the heart, decreases outward potassium current I_{K1}, and/or increases inward current I_{f}.

Further, the biological pacemaker may be provided to the Bachman's bundle, sinoatrial node, atrioventricular junctional region, His branch, left or right bundle branch, Purkinke fibers, right or left atrial muscle or ventricular muscle of the subject's heart.

Cardiac rhythm disorders that may be treated using the tandem systems of the invention include, for example, sinus node dysfunction, sinus bradycardia, marginal pacemaker activity, sick sinus syndrome, tachyarrhythmia, sinus node reentry tachycardia, atrial tachycardia from an ectopic focus, atrial flutter, atrial fibrillation, bradyarrhythmia, or cardiac failure and wherein the biological pacemaker is administered to the left or right atrial muscle, sinoatrial node, or atrioventricular junctional region of the subject's heart.

In an embodiment of the invention, the electronic pacemaker is programmed to sense the subject's heart beating rate and to produce a pacemaker signal when the heart beating rate falls below a selected heart beating rate. The selected beating rate is a selected proportion of the beating rate experienced by the heart in a reference time interval. The reference time interval is an immediately preceding time period of selected duration.

The present invention provides a method of treating a cardiac rhythm disorder, wherein the disorder is a conduction block, complete atrioventricular block, incomplete atrioventricular block, bundle branch block, cardiac failure, or a bradyarrhythmia, the method comprising administering a tandem pacemaker system comprising a bypass tract and an electronic pacemaker to the subject's heart such that the bypass tract spans the region exhibiting defective conductance, wherein transmission by the bypass tract of an electronic pacemaker current induced by the electronic pacemaker is effective to treat the subject, and wherein the electronic pacemaker is provided either prior to, simultaneously with or after the bypass tract is provided.

The present invention is also directed to a method of treating a subject afflicted with a sinus node dysfunction, sinus bradycardia, marginal pacemaker activity, sick sinus syndrome, cardiac failure, tachyarrhythmia, sinus node reentry tachycardia, atrial tachycardia from an ectopic focus, atrial flutter, atrial fibrillation, or a bradyarrhythmia and a conduction block disorder, which method comprises administering a tandem pacemaker system comprising a biological pacemaker, a bypass tract and an electronic pacemaker, wherein an electronic pacemaker is provided either prior to, simultaneously with, or after the biological pacemaker is provided, and wherein the biological pacemaker is administered to the subject to generate an effective biological pacemaker current in the subject's heart, and wherein a bypass tract spans the region exhibiting defective conduction, wherein transmission by the bypass tract of an electronic pacemaker and/or biological pacemaker current is effective to treat the subject.

The present invention further relates to a method of treating a subject afflicted with ventricular dyssynchrony comprising (a) selecting a site in a first ventricle of the subject's heart, (b) administering a biological pacemaker of as described herein to the selected site so as to initiate pacemaker activity and stimulate contraction of the first ventricle, and (c) pacing a second ventricle of the heart with a first electronic pacemaker which is programmed to detect a signal from the biological pacemaker and to produce a pacemaker signal at a reference time interval after the biological pacemaker signal is detected, thereby providing biventricular pacemaker function to treat the subject.

In a specific embodiment, the electronic pacemaker is further programmable to produce a pacemaker signal when it fails to detect a signal from the biological pacemaker after a time period of specified duration. Additionally, the system may further comprise a second electronic pacemaker to be administered to a coronary vein, wherein the second electronic pacemaker is programmable to detect a signal from the biological pacemaker and to produce a pacemaker signal in tandem with the first electronic pacemaker if said second electronic pacemaker fails to detect a signal from the biological pacemaker after a time period of specified duration, the first and second electronic pacemakers thereby providing biventricular function.

A tandem pacemaker system for treating a subject afflicted with ventricular dyssynchrony is provided comprising (1) a biological pacemaker to be administered to a first ventricle of the subject's heart, and (2) an electronic pacemaker to be administered to a second ventricle of the subject's heart, wherein the electronic pacemaker is programmable to detect a signal from the biological pacemaker and to produce a electronic pacemaker signal at a reference time interval after the biological pacemaker signal is detected, so as to thereby provide biventricular pacemaker function, and wherein the electronic pacemaker is provided either prior or simultaneously with the biological pacemaker.

Such a pacemaker system may further comprise a second electronic pacemaker to be administered to a coronary vein, wherein the second electronic pacemaker is programmable to detect a signal from the biological pacemaker and to produce a pacemaker signal in tandem with the first electronic pacemaker if said second electronic pacemaker fails to detect a signal from the biological pacemaker after a time period of specified duration, the first and second electronic pacemakers thereby providing biventricular function.

### Uses and Administration of the Compositions of the Invention

The present invention provides methods and compositions which may be used for treatment of various diseases associated with cardiac rhythm disorders. Cardiac rhythm disorders that may be treated include pathological arrhythmia, conduction block, complete atrioventricular block, incomplete atrioventricular block, bundle branch block, weak pacemaker activity, sinus node dysfunction, sinus bradycardia, sick sinus syndrome, bradyarrhythmia, tachyarrhythmia, Sinoatrial nodal re-entry tachycardia, atrial tachycardia from an ectopic focus, atrial flutter, atrial fibrillation, or cardiac failure.

The methods of the invention, comprise administration of late passage MSCs in a pharmaceutically acceptable carrier, for treatment of cardiac disorders. “Administering” shall mean delivering in a manner which is effected or performed using any of the various methods and delivery systems known to those skilled in the art. Administering can be performed, for example, pericardially, intracardially, subepicardially, transendocardially, via implant, via catheter, intracoronarily, intravenously, intramuscularly, subcutaneously, parenterally, topically, orally, transmucosally, transdermally, intradermally, intraperitoneally, intrathecally, intralymphatically, intralesionally, epidurally, or by in vivo electroporation. Administering can also be performed, for example, once, a plurality of times, and/or over one or more extended periods.

Cell-based biological pacemaker may require focal delivery. Several methods to achieve focal delivery are feasible; for example, the use of catheters and needles, and/or growth on a matrix and a “glue.” Whatever approach is selected, the delivered cells should not disperse from the target site. Such dispersion could introduce unwanted electrical effects within the heart or in other organs.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition can be formulated as a suppository, with traditional binders and carriers such as triglycerides. Oral formulation can include standard carvers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical sciences” by E. W. Martin. Such compositions will contain a therapeutically effective amount of the therapeutic compound, preferably in purified form, together with a suitable amount of carrier so as to provide the form for proper administration to the patient. The formulation should suit the mode of administration.

The appropriate concentration of the composition of the invention which will be effective in the treatment of a particular cardiac disorder or condition will depend on the nature of the disorder or condition, and can be determined by one of skill in the art using standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses maybe extrapolated from dose response curves derived from in vitro or animal model test systems. Additionally, the administration of the compound could be combined with other known efficacious drugs if the in vitro and in vivo studies indicate a synergistic or additive therapeutic effect when administered in combination.

The progress of the recipient receiving the treatment may be determined using assays that are designed to test cardiac function. Such assays include, but are not limited to ejection fraction and diastolic volume (e.g., echocardiography), PET scan, CT scan, angiography, 6-minute walk test, exercise tolerance and NYHA classification.

### EXAMPLE

### Biological Features of Late Passage Mesenchymal Stem Cells

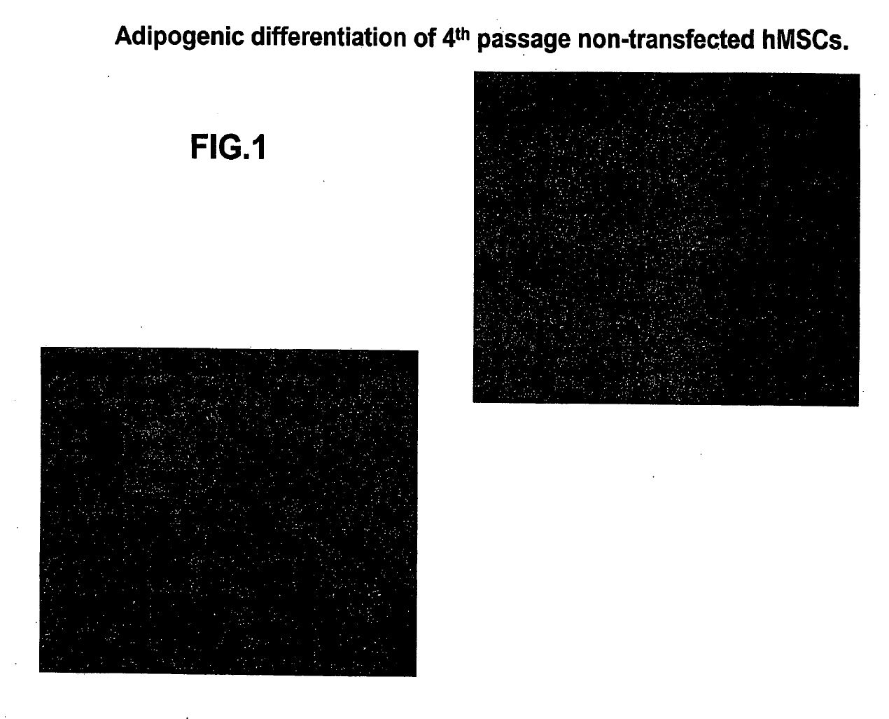

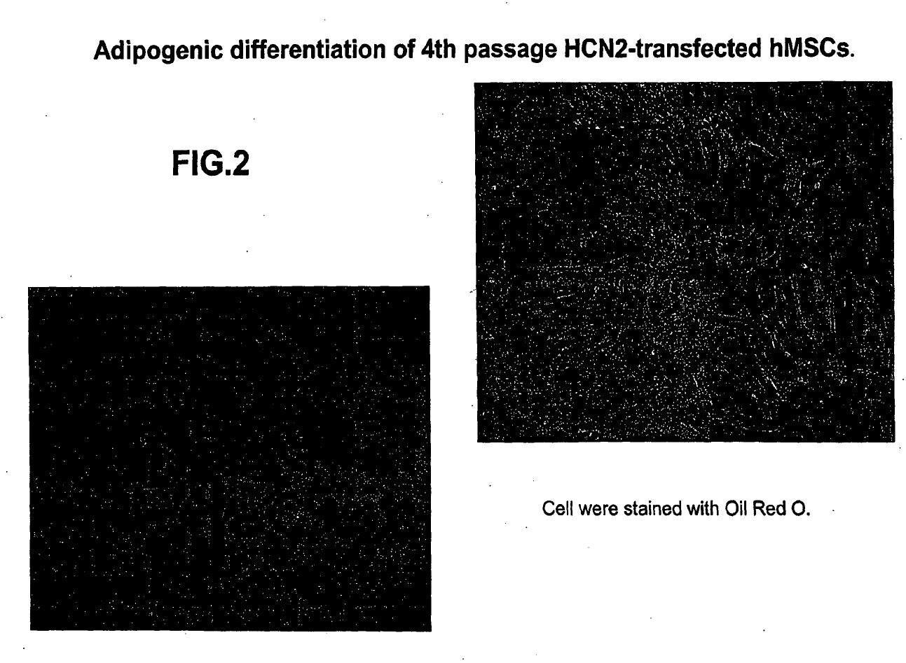

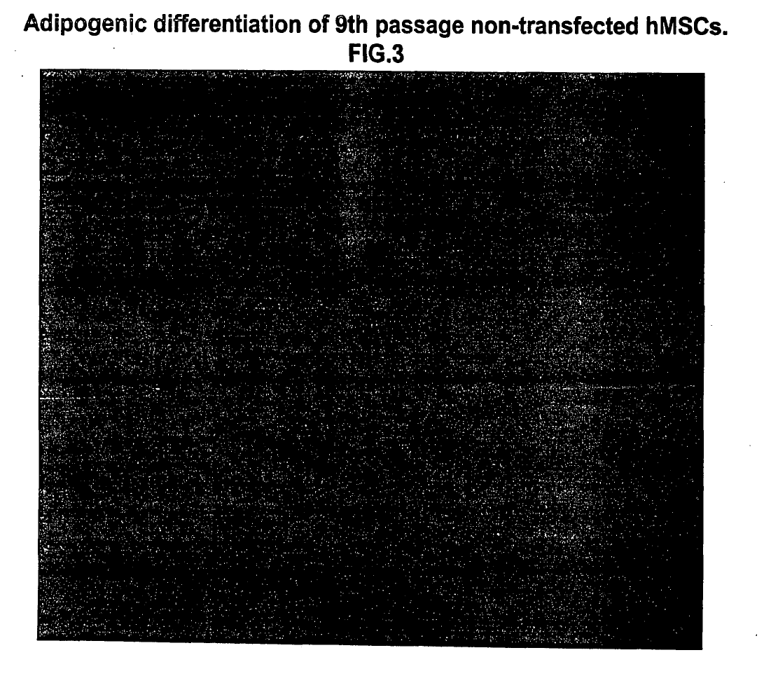

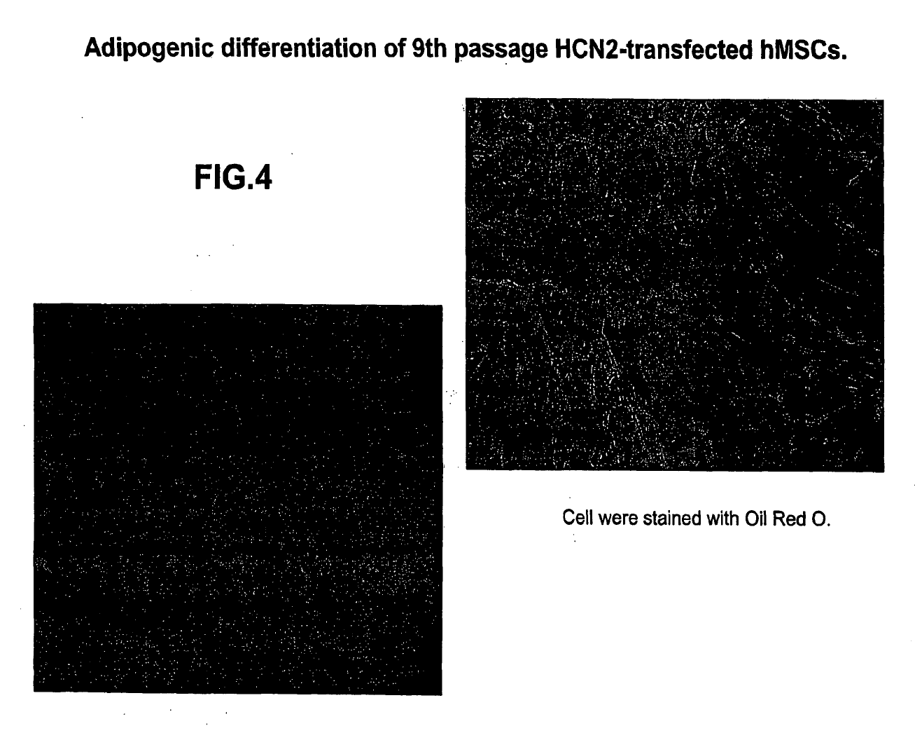

Experiments were performed to determine the biological features of late passage MSCs. hMSCs were purchased and thawed, subcultured and maintained according to the supplier's directions (Cambrex Corporation.). As demonstrated in FIG. 1, fat vacuoles are observed in 4^{th} passage hMSCs exposed to adipogenic differentiation using a purchased kit and the manufacturer's directions (see instructions for adipogenic assay procedure from Cambrex Corporation). In 4^{th} passage hSCs first transfected with the PIRES-HCN2 plasmid followed by exposure to adipogenic differentiation, fewer cells with fat vacuoles were observed, but staining with oil red O still demonstrates a significant number of positive (red) cells (FIG. 2). See instructions for oil red O staining for in vitro adipogenesis from Cambrex Corporation. In contrast, minimal adipogenic differentiation of 9^{th} passage non-transfected hMSCs is demonstrated by the presence of few fat vacuoles (FIG. 3). FIG. 4 indicates the absence of adipogenic differentiation in 9^{th} passages hMSCs transfected with the PIRES-HCN2 plasmid.

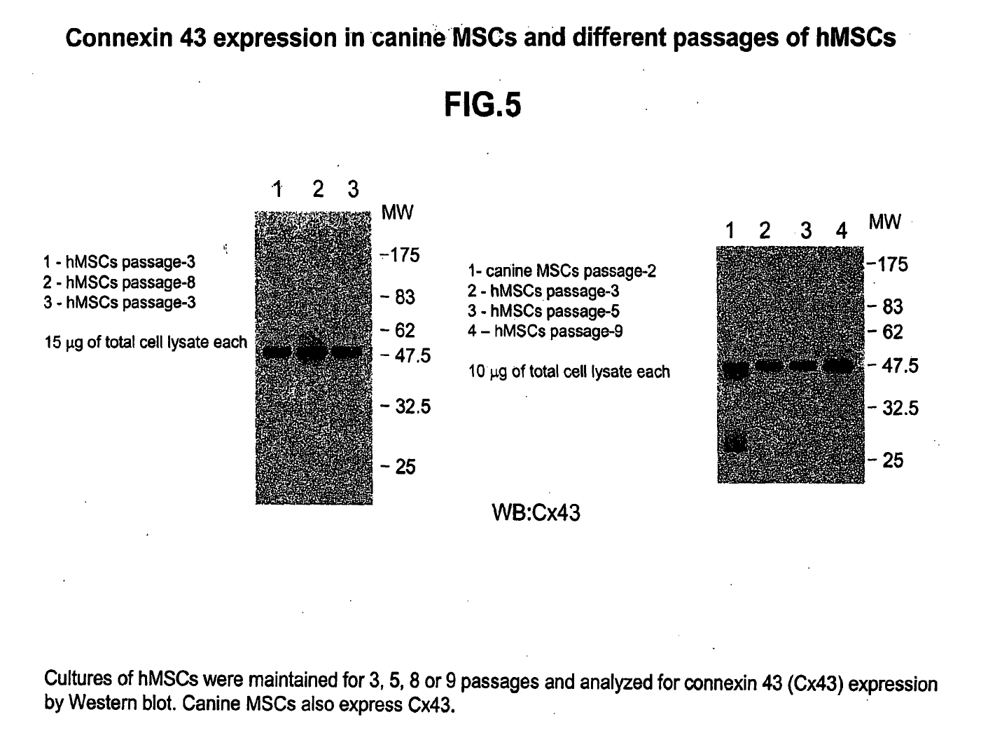

FIG. 5 depicts Western blots demonstrating abundant connexin 43 expression in 3^{rd} and 8^{th} passage hMSCs (right panel) and 3, 5 and 9^{th} passage hMSCs and 2^{nd} passage canine hMSCs (right panel).

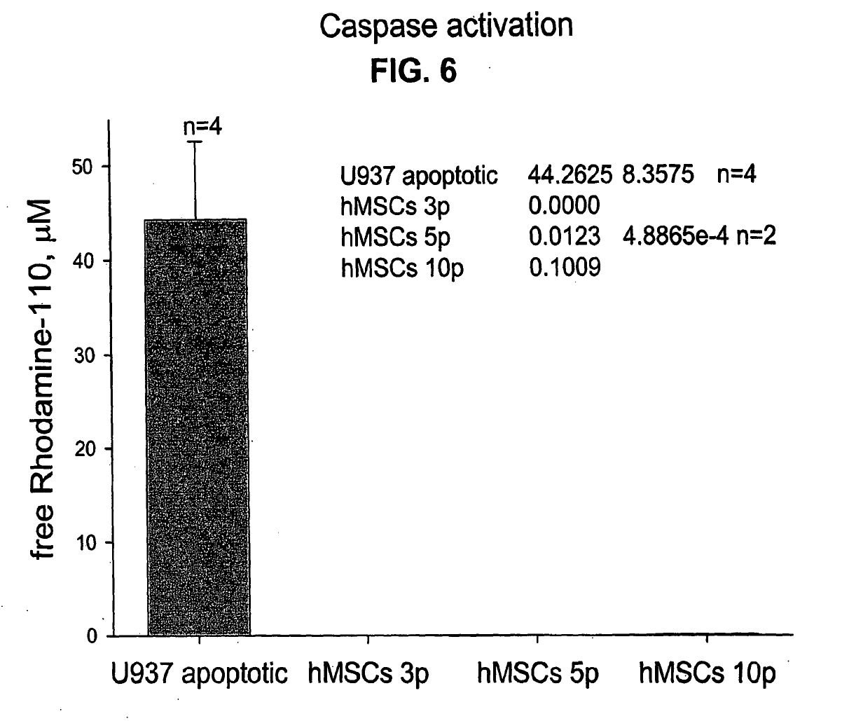

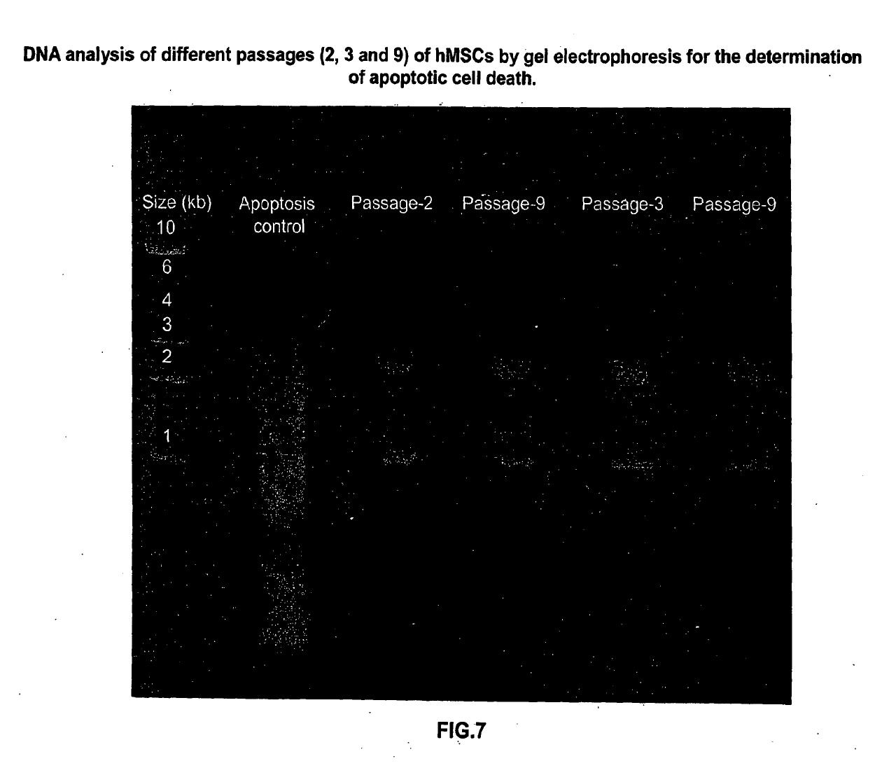

To determine the predisposition of late passage MSCs to apoptosis, caspase activation was assayed for. FIG. 6 demonstrates minimal activation for hMSCs at passages 3, 5 or 10 indicating no predisposition to apoptosis. Additionally, as depicted in FIG. 7. there is no DNA fragmentation, further indicating that these passaged hMSCs do not have a predisposition to apoptosis.

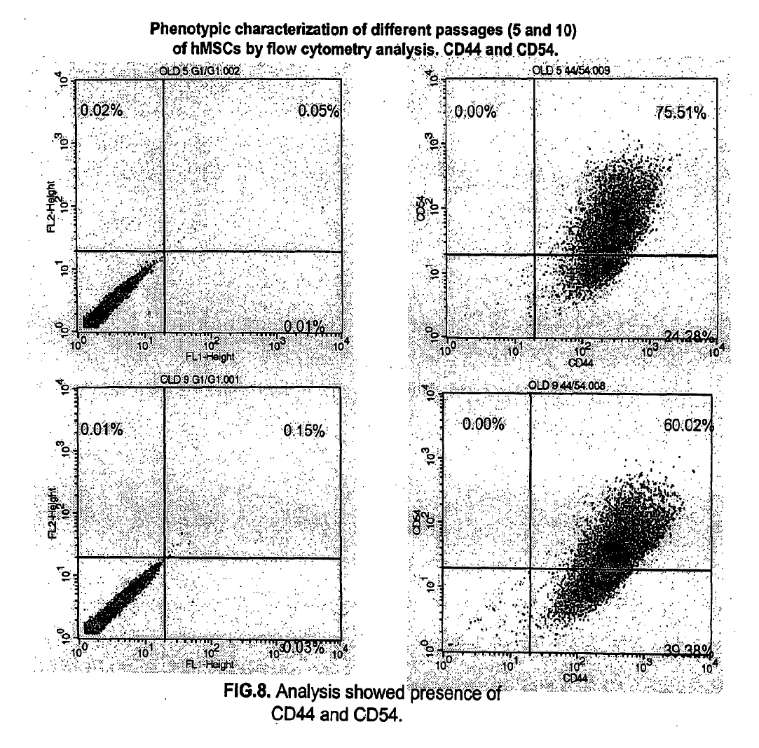

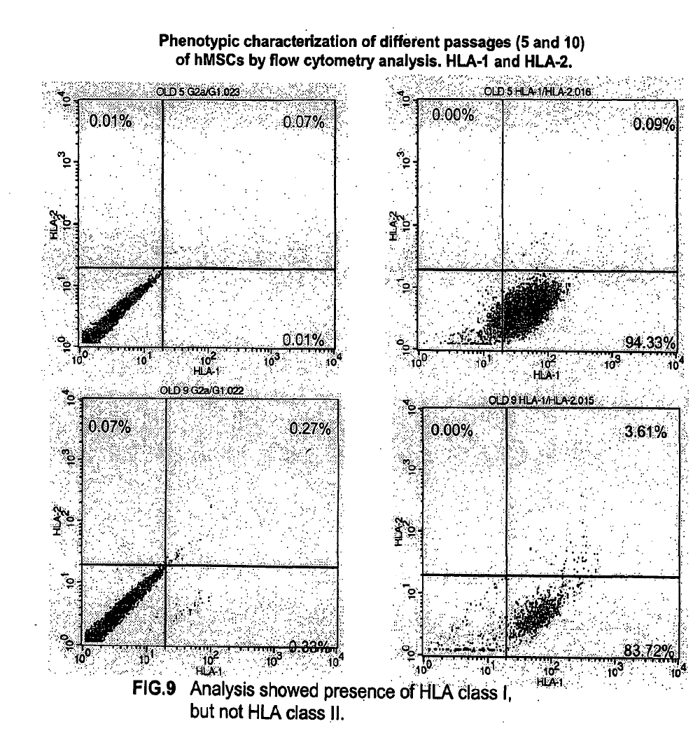

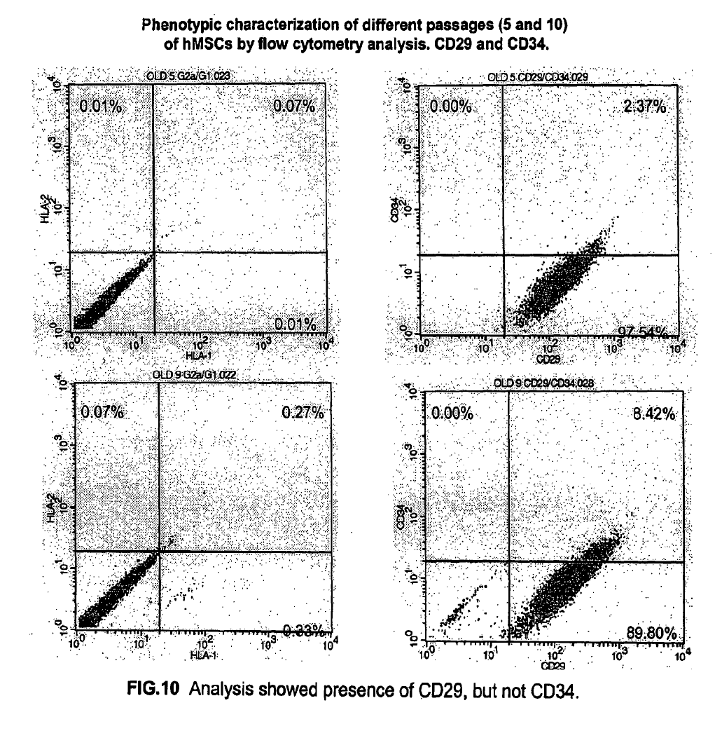

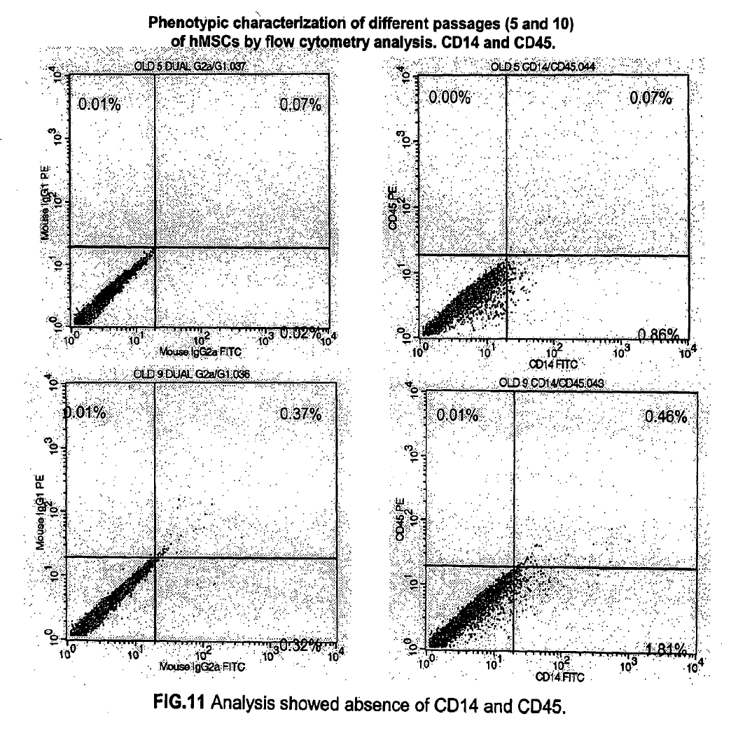

Phenotypic characterization of cell surface antigen expression was examined on late passage MSCs by flow cytometry. The results indicate the presence of CD44 and CD54 antigen (FIG. 8), the presence of HLA I markers but not HLA class II markers (FIG. 9) and the presence of CD29 but not CD34 in both passage 5 and 10 cells. FIG. 11 demonstrates the absence of CD14 and CD45 antigens in both sets of cells.

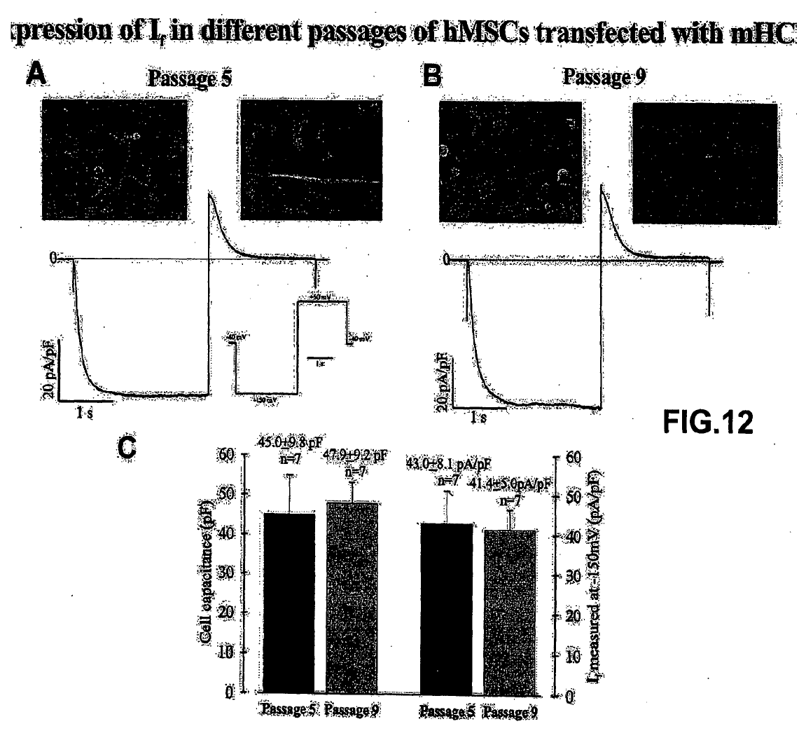

FIG. 12 demonstrates that expression of HCN2-induced Ir like current is the same in cells from passages 5 and 9 transfected with the PIRES-HCN2 plasmid: FIG. 12A depicts fluorescence images of passage 5 cells (upper two panels) and sample current record from patch clamp recordings (lower panel). FIG. 12B depicts fluorescence images of passage 9 cells (upper 2 panels) and sample current record from patch clamp recordings (lower panel); FIG. 12C is a histogram comparing the capacitance (left 2 bars) and the HCN2-induced current density (right two bars). There is no significant difference in either parameter between hMSCs from passage 5 and 9.

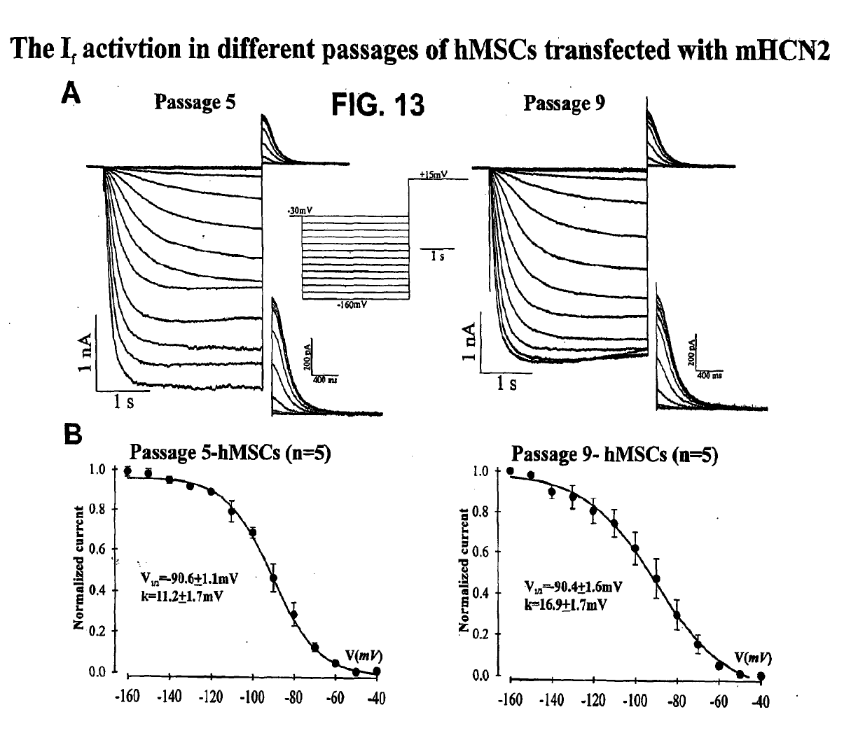

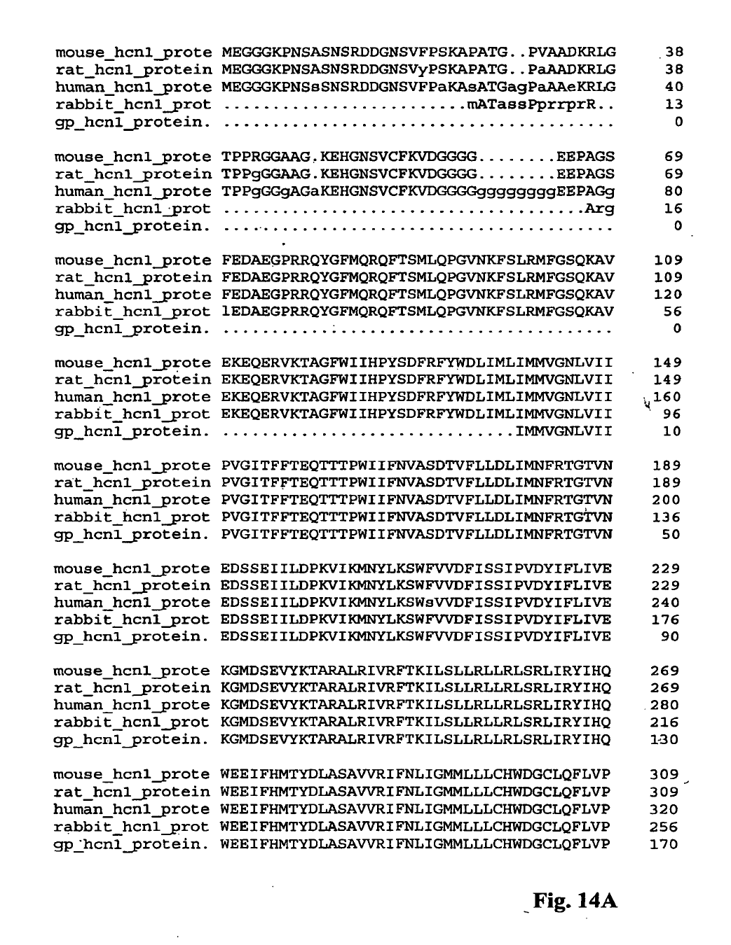

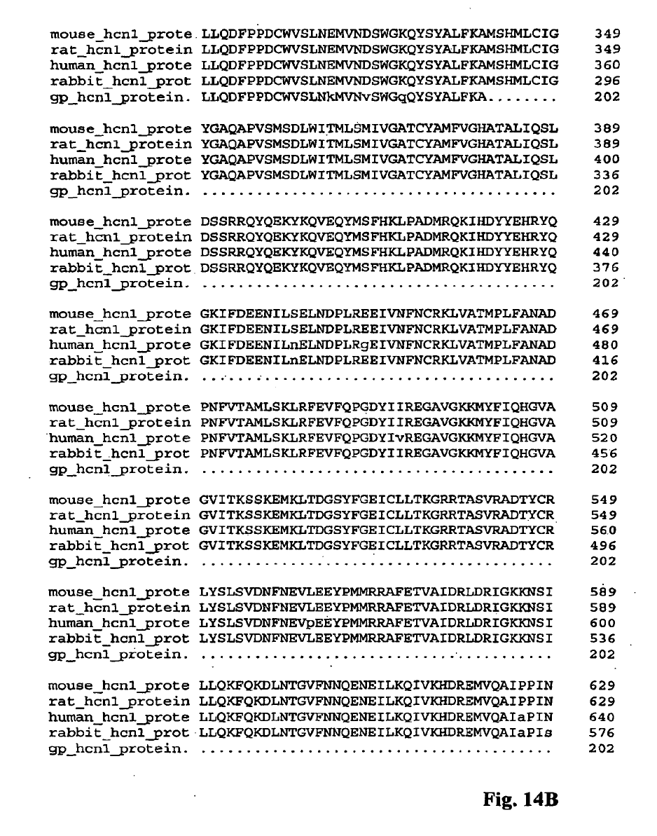

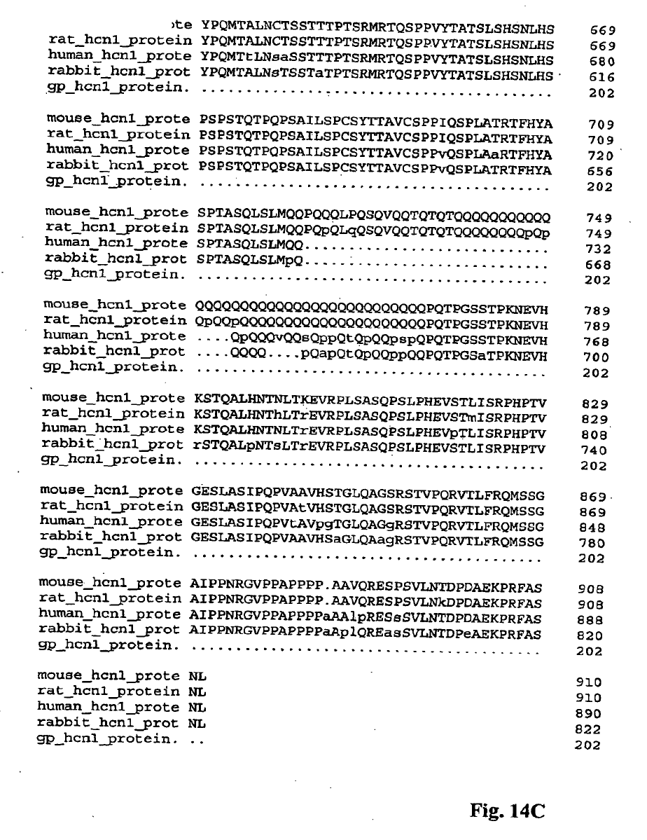

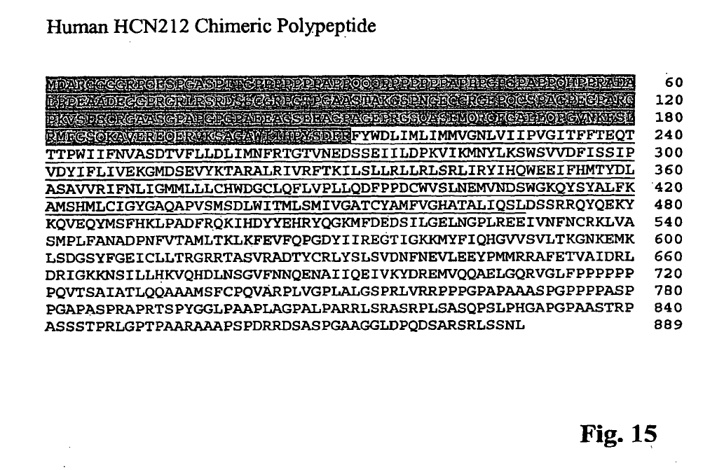

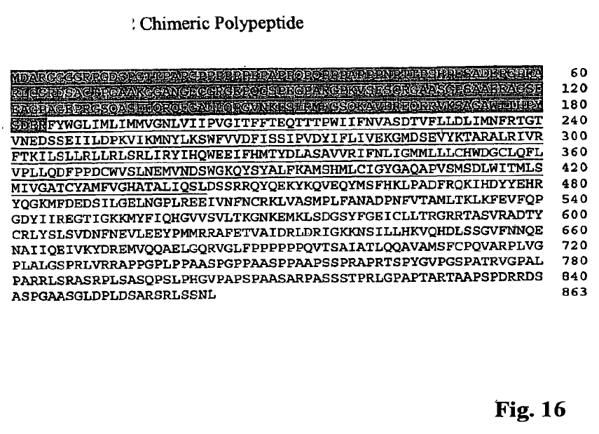

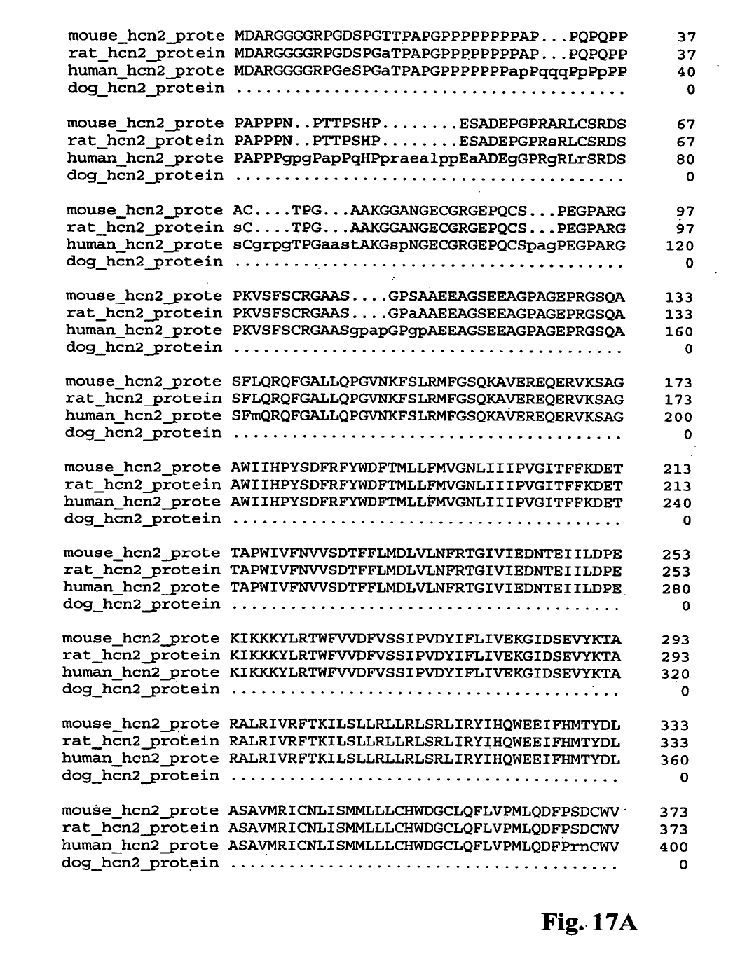

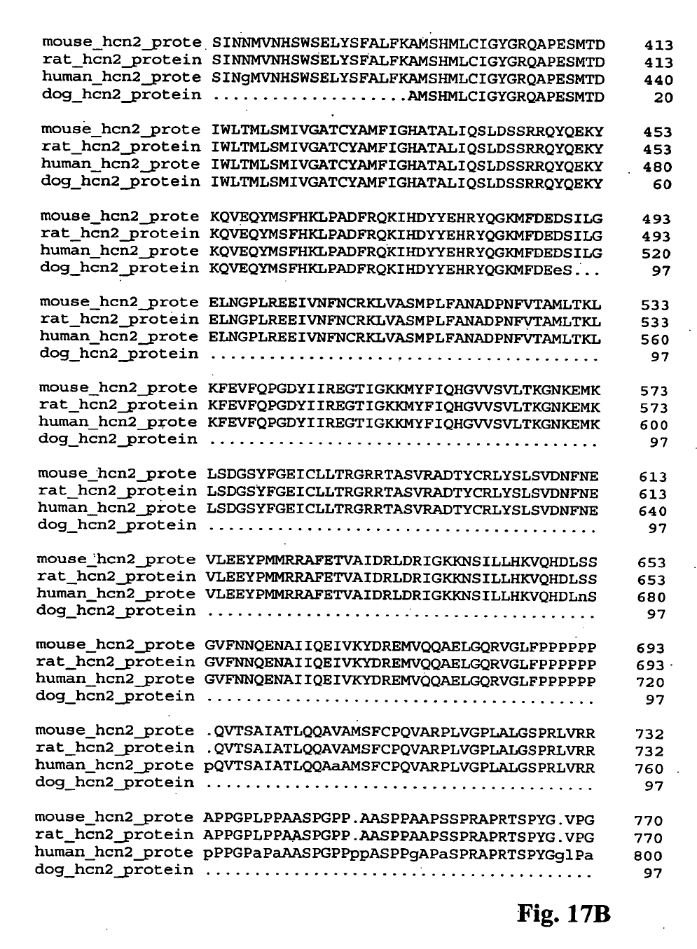

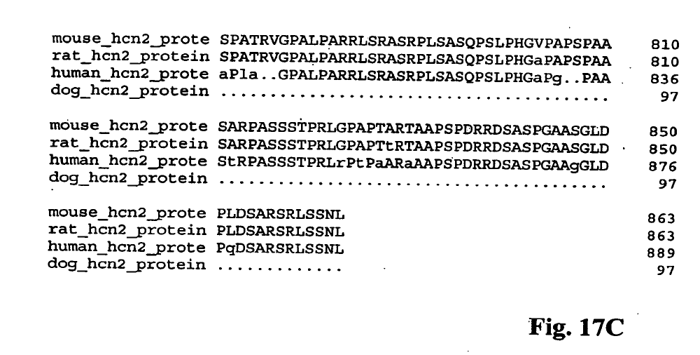

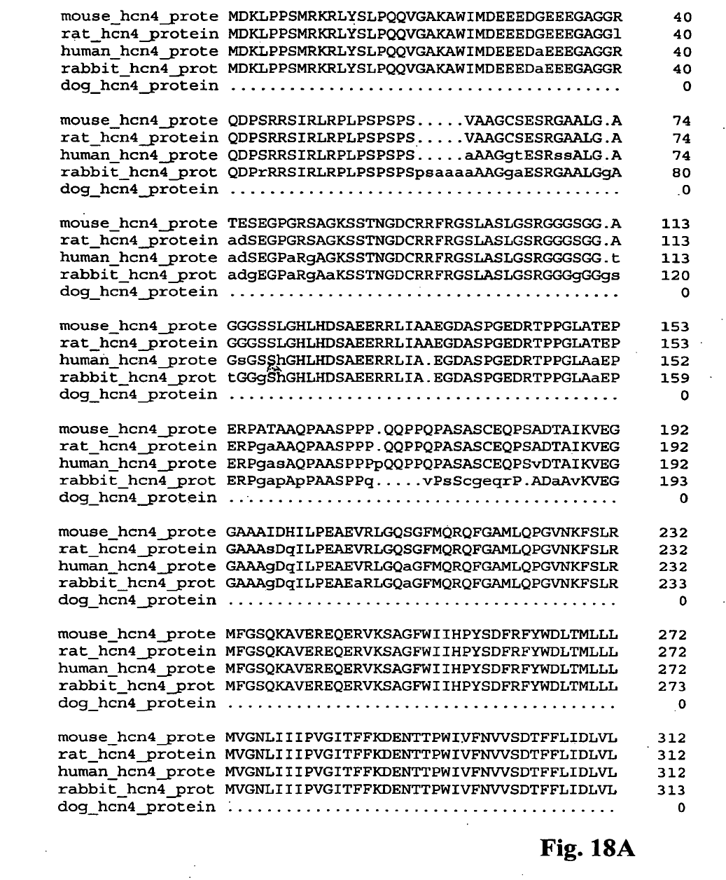

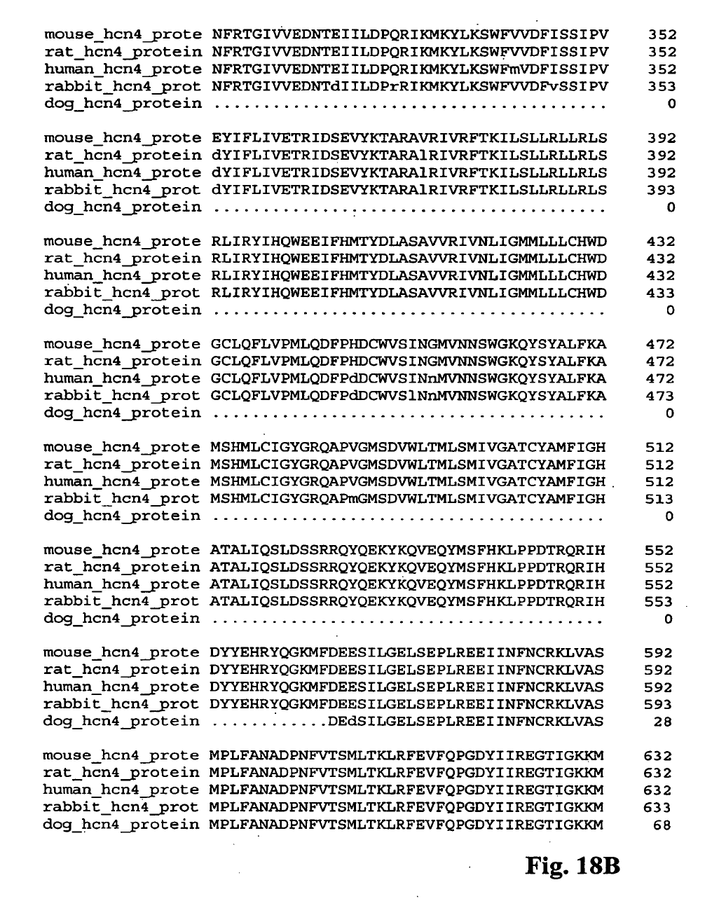

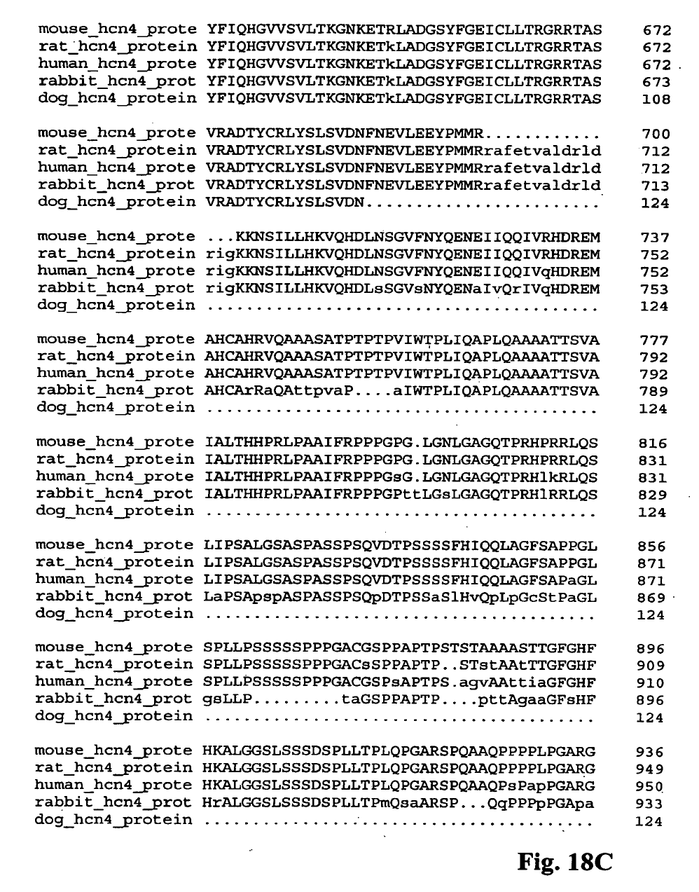

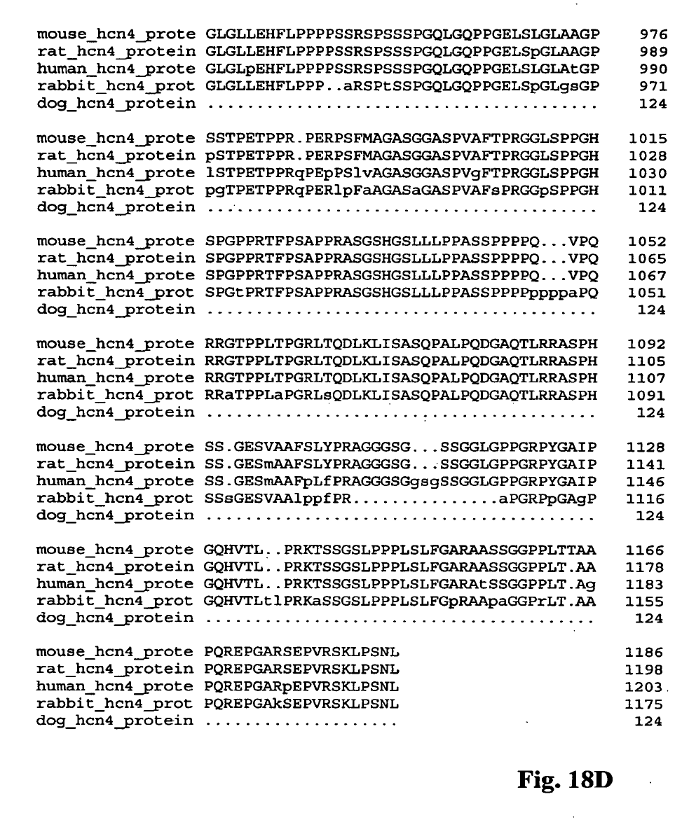

FIG. 13 demonstrates that the biophysical properties of passage 5 and passage 9 cells expressing HCN2-induced current are very similar. FIG. 13A is a comparison of current records of HCN2-included current in passage 5 (left panel) and passage 9 (right panel) hMSCs. The current records are very similar. FIG. 13B depicts activation curves obtained from passage 5 (left panel) and passage 9 (right panel) cells show the same midpoint of activation.

1-27. (canceled)

28. An isolated human adult mesenchymal stem cell, which has been passaged at least nine times, and which functionally expresses a hyperpolarization-activated, cyclic nucleotide-gated (HCN) ion channel, and wherein expression of the HCN channel is effective to induce a pacemaker current in said cell.

29. The mesenchymal stem cell of claim 28, which
(i) expresses CD29, CD44, CD54 and HLA class I surface markers; and
(ii) do not express CD14, CD45, CD34 and HLA class II surface markers.

30. The human adult mesenchymal stem cell of claim 28 wherein said cell functionally expresses a MiRP1 beta subunit.

31. The human adult mesenchymal stem cell of claim 28, wherein the HCN ion channel is a (i) mutant HCN channel; or a (ii) chimeric HCN channel comprising an amino terminal portion, an intramembrane portion, and a carboxy terminal portion, wherein the portions are derived from more than one HCN isoform.

32. A pharmaceutical composition comprising the population of human adult mesenchymal stem cells of claim 28 and a pharmaceutically acceptable carrier.

33. The pharmaceutical composition of claim 32, wherein said cell functionally expresses a MiRP1 beta subunit.

34. The pharmaceutical composition of claim 32, wherein the HCN ion channel is a (i) mutant HCN channel; or a (ii) chimeric HCN channel comprising an amino terminal portion, an intramembrane portion, and a carboxy terminal portion, wherein the portions are derived from more than one HCN isoform.

35. The pharmaceutical composition of claim 32, wherein the population of human adult mesenchymal stem cells:
(i) expresses CD29, CD44, CD54 and HLA class I surface markers; and
(ii) do not express CD14, CD45, CD34 and HLA class II surface markers.

36. The pharmaceutical composition of claim 32, comprising an amount of mesenchymal stem cells sufficient to generate biological pacemaker activity in a subject.

37. An atrioventricular (AV) bridge comprising gap junction-coupled human adult mesenchymal stem cells, which have been passaged at least nine times, the bridge having a first end and a second end, both ends capable of being attached to two selected sites in a heart, so as to allow the propagation of an electrical signal across a tract between the two sites in the heart.

38. The AV bridge of claim 37, wherein the first end is capable of being attached to the atrium and the second end capable of being attached to the ventricle, so as to allow propagation of an electrical signal from the atrium to travel across the tract to excite the ventricle.

39. The AV bridge of claim 37, wherein the human adult mesenchymal stem cells:
(i) expresses CD29, CD44, CD54 and HLA class I surface markers; and
(ii) do not express CD14, CD45, CD34 and HLA class II surface markers.

40. The AV bridge of claim 37, wherein the cells of the tract functionally express at least one protein selected from the group consisting of: a connexin; an alpha subunit and accessory subunits of a L-type calcium channel; an alpha subunit with or without the accessory subunits of a sodium channel; and a L-type calcium and/or sodium channel in combination with the alpha subunit of a potassium channel, with or without the accessory subunits of the potassium channel.

41. The AV bridge of claim 37 wherein said human adult mesenchymal stem cells are transfected to express:
(a) a hyperpolarization-activated, cyclic nucleotide-gated (HCN) ion channel capable of generating a pacemaker current is said cell, or
(b) a chimeric HCN channel comprising an amino terminal portion, an intramembrane portion, and a carboxy terminal portion, wherein the portions are derived from more than one HCN isoform, and wherein the expressed chimeric HCN channel generates a pacemaker current in said cell, or
(c) a mutant HCN channel wherein the mutant HCN channel generates a pacemaker current in said cell.

42. The AV bridge of claim 41, wherein said human adult mesenchymal stem cells functionally express a MiRP1 beta subunit.

43. A method for generating biological pacemaker activity in a subject, comprising administering to said subject an effective amount of isolated human adult mesenchymal stem cells, which has been passaged at least nine times, and which functionally expresses a hyperpolarization-activated, cyclic nucleotide-gated (HCN) ion channel, and wherein expression of the HCN channel is effective to induce biological pacemaker activity in said cell.

44. The method of claim 43 wherein the mesenchymal stem cell
(i) expresses CD29, CD44, CD54 and HLA class I surface markers; and
(ii) do not express CD14, CD45, CD34 and HLA class II surface markers.

45. The method of claim 43, wherein the human adult mesenchymal stem cells functionally express:
(a) a hyperpolarization-activated, cyclic nucleotide-gated (HCN) ion channel capable of generating a pacemaker current in said mesenchymal stem cells,
(b) a chimeric HCN channel comprising an amino terminal portion, an intramembrane portion, and a carboxy terminal portion, wherein the portions are derived from more than one HCN isoform, and wherein the expressed chimeric HCN channel generates a pacemaker current in said human adult mesenchymal stem cells, or
(c) a mutant HCN channel wherein the mutant HCN channel generates a pacemaker current in said human adult mesenchymal stem cells.

46. The method of claim 45, wherein said human adult mesenchymal stem cells functionally expresses a MiRP1 beta subunit.

47. The method of claim 43, wherein said subject is afflicted with a cardiac rhythm disorder.

48. The method of claim 43, wherein said subject is afflicted with a disorder at the sino-atrial node.

49. The method of claim 43, wherein the said subject is afflicted with a disorder of the atrioventricular node.

50. The method of claim 43, wherein expression of the HCN channel is effective to treat a cardiac rhythm disorder.

51. The method of claim 43, wherein expression of the HCN channel is effective to inhibit the onset of a cardiac rhythm disorder.

52. The method of claim 50 or 51, wherein the cardiac rhythm disorder is selected from the group consisting of sinus node dysfunction, sinus bradycardia, marginal pacemaker function, sick sinus syndrome, tachyarrhythmia, sinus node reentry tachycardia, atrial tachycardia, atrial flutter, atrial fibrillation, bradyarrhythmia, cardiac failure, conduction block, complete atrioventricular block, incomplete atrioventricular block or bundle branch block.

53. A method of treating a subject afflicted with a disorder of the atrioventricular node, comprising:
(i) administering an atrioventricular (AV) bridge to the subject having a disorder of the atrioventricular node, said bridge comprising gap junction-coupled human adult mesenchymal stem cells, which have been passaged at least nine times, the bridge having a first end and a second end, both ends capable of being attached to two selected sites in a heart, and
(ii) attaching both ends of bridge to two selected sites in the heart, so as to allow the propagation of an electrical signal across a tract between the two sites in the heart.

54. The method of claim 53, wherein the first end is capable of being attached to the atrium and the second end is capable of being attached to the ventricle, so as to allow propagation of an electrical signal from the atrium to travel across the tract to excite the ventricle.

55. The method of claim 53 wherein the mesenchymal stem cell
(i) expresses CD29, CD44, CD54 and HLA class I surface markers; and
(ii) do not express CD14, CD45, CD34 and HLA class II surface markers.

56. The method of claim 55, wherein the human adult mesenchymal stem cells functionally express:
(a) a hyperpolarization-activated, cyclic nucleotide-gated (HCN) ion channel capable of generating a pacemaker current in said mesenchymal stem cells,
(b) a chimeric HCN channel comprising an amino terminal portion, an intramembrane portion, and a carboxy terminal portion, wherein the portions are derived from more than one HCN isoform, and wherein the expressed chimeric HCN channel generates a pacemaker current in said human adult mesenchymal stem cells, or
(c) a mutant HCN channel wherein the mutant HCN channel generates a pacemaker current in said human adult mesenchymal stem cells.

57. The method of claim 56, wherein said human adult mesenchymal stem cells functionally expresses a MiRP1 beta subunit.

58. The method of claim 53, wherein the human adult mesenchymal stem cells functionally express at least one protein selected from the group consisting of: a connexin; an alpha subunit and accessory subunits of a L-type calcium channel; an alpha subunit with or without the accessory subunits of a sodium channel; and a L-type calcium and/or sodium channel in combination with the alpha subunit of a potassium channel, with or without the accessory subunits of the potassium channel.

59. A tandem pacemaker system comprising:
(1) an electronic pacemaker; and
(2) a biological pacemaker comprising comprises implantable human adult mesenchymal stem cells, which have been passaged at least nine times, that functionally expresses a (a) an HCN ion channel, or (b) a chimeric HCN channel wherein the chimeric HCN channel comprises portions of more than one type of HCN channel, or (c) a mutant HCN channel;
wherein the expressed HCN channel generates an effective pacemaker current when the cell is implanted into a subject's heart.

60. A tandem pacemaker system comprising:
(1) an electronic pacemaker;
(2) a bypass bridge comprising a strip of gap junction-coupled human adult mesenchymal stem cells, which have been passaged at least nine times, having a first end and a second end, both ends capable of being attached to two selected sites in a heart, so as to allow the transmission of a pacemaker and/or electrical signal/current across the tract between the two sites in the heart; and
(3) a biological pacemaker comprising an implantable late passage mesenchymal stem cell that functionally expresses a (a) an HCN ion channel, or (b) a chimeric HCN channel wherein the chimeric HCN channel comprises portions of more than one type of HCN channel, or (c) a mutant HCN channel;
wherein the expressed HCN, chimeric HCN or mutant HCN channel generates an effective pacemaker current when said cell is implanted into a subject's heart.

61. The use of claim 59 or 60 to treat a cardiac rhythm disorder, wherein the biological pacemaker of the system is provided to the subject's heart to generate biological pacemaker activity and the electronic pacemaker is provided to work in tandem with the biological pacemaker to treat the cardiac rhythm disorder.

62. The tandem pacemaker system of claim 59 or 60 wherein the mesenchymal stem cell;
(i) expresses CD29, CD44, CD54 and HLA class I surface markers; and
(ii) do not express CD14, CD45, CD34 and HLA class II surface markers.
